(19) United States
(12) Patent Application Publication
(10) Pub. No.: US 2018/0098959 A1
(43) Pub. Date: Apr. 12, 2018

(54) METHODS FOR TREATING CANCER

(71) Applicant: BOSTON BIOMEDICAL, INC., Cambridge, MA (US)

(72) Inventors: Chiang J. LI, Cambridge, MA (US); Youzhi LI, Westwood, MA (US); Laura BORODYANSKY, Brookline, MA (US); Yuan GAO, Belmont, MA (US); David LEGGETT, Milton, MA (US); David P. KERSTEIN, North Reading, MA (US)

(73) Assignee: BOSTON BIOMEDICAL, INC., Cambridge, MA (US)

(21) Appl. No.: 15/567,070

(22) PCT Filed: Apr. 18, 2016
(86) PCT No.: PCT/US2016/028179

§ 371 Date: Oct. 16, 2017

Related U.S. Application Data
(60) Provisional application No. 62/149,349, filed on Apr. 17, 2015.

(51) Int. Cl. A61K 31/343; A61P 35/00; C07K 16/28; A61K 31/7068; A61K 31/44; A61K 31/519; A61K 31/513; A61K 31/282; C07K 16/22; A61P 35/04; A61K 39/395

(52) U.S. Cl. CPC A61K 31/343; A61P 35/00; C07K 16/2863; A61K 31/7068; A61K 31/44; A61K 31/519; C07K 2317/24; A61K 31/282; C07K 16/22; A61P 35/04; A61K 39/3955; C07K 2317/21; A61K 31/513

(57) ABSTRACT

Methods comprising administration of and kits comprising at least one compound of formula (I); and at least one additional anti-cancer therapy chosen from panitumumab, cetuximab, capecitabine, CAPDX, regorafenib, and FOLFOX.

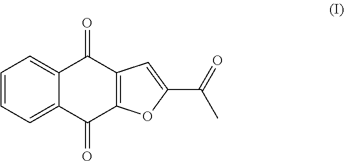 [SMILES: CC(=O)c1cc2c(o1)C(=O)c1ccccc1C2=O]

The present application claims the benefit of priority under 35 U.S.C. § 119 of U.S. Provisional Patent Application No. 62/149,349, filed Apr. 17, 2015, the contents of which was incorporated herein by reference.

Disclosed herein are methods comprising administering to a subject a combination comprising a therapeutically effective amount of at least one compound of formula (I) in combination with a therapeutically effective amount of: (i) at least one panitumumab compound chosen from panitumumab, pharmaceutically acceptable salts thereof, and solvates of any of the foregoing; (ii) at least one cetuximab compound chosen from cetuximab, pharmaceutically acceptable salts thereof, and solvates of any of the foregoing; (iii) at least one leucovorin compound chosen from leucovorin, pharmaceutically acceptable salts thereof, and solvates of any of the foregoing, at least one oxaliplatin compound chosen from oxaliplatin, pharmaceutically acceptable salts thereof, and solvates of any of the foregoing, and at least one 5-fluorouracil compound chosen from 5-fluorouracil, pharmaceutically acceptable salts thereof, and solvates of any of the foregoing (the combination of which components will be referred to as “FOLFOX” as defined below), optionally in combination with at least one angiogenesis inhibitor; (iv) at least one capecitabine compound chosen from capecitabine, pharmaceutically acceptable salts thereof, and solvates of any of the foregoing optionally in combination with at least one oxaliplatin compound chosen from oxaliplatin, pharmaceutically acceptable salts thereof, and solvates of any of the foregoing (the combination of which components will be referred to as “CAPDX” as defined below); or (v) at least one regorafenib compound chosen from regorafenib, pharmaceutically acceptable salts thereof, and solvates of any of the foregoing.

The at least one compound of formula (I) is chosen from compounds having formula (I)

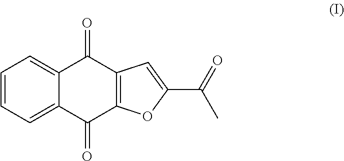 [SMILES: CC(=O)c1cc2c(o1)C(=O)c1ccccc1C2=O]

prodrugs, derivatives, pharmaceutically acceptable salts of any of the foregoing, and solvates of any of the foregoing.

Cancer fatalities in the United States alone number in the hundreds of thousands each year. Despite advances in the treatment of certain forms of cancer through surgery, radiotherapy, and chemotherapy, many types of cancer are essentially incurable. Even when an effective treatment is available for a particular cancer, the side effects of such treatment can be severe and result in a significant decrease in quality of life.

Most conventional chemotherapy agents have toxicity and limited efficacy, particularly for patients with advanced solid tumors. Conventional chemotherapeutic agents cause damage to non-cancerous as well as cancerous cells. The therapeutic index (i.e., a measure of a therapy's ability to discriminate between cancerous and normal cells) of such chemotherapeutic compounds can be quite low. Frequently, a dose of a chemotherapy drug that is effective to kill cancer cells will also kill normal cells, especially those normal cells (such as epithelial cells and cells of the bone marrow) that undergo frequent cell division. When normal cells are affected by the therapy, side effects such as hair loss, suppression of hematopoiesis, and nausea can occur. Depending on the general health of a patient, such side effects can preclude the administration of chemotherapy, or, at least, be extremely unpleasant and uncomfortable for the patient and severely decrease quality of the remaining life of cancer patients. Even for cancer patients who respond to chemotherapy with tumor regression, cancers often quickly relapse, progress and form more metastasis after initial response to chemotherapy. Such recurrent cancers become highly resistant or refractory to chemotherapeutics. As discussed below, cancer stem cells (CSCs) or cancer cells with high stemness (stemness-high cancer cells) are responsible for the rapid tumor recurrence and resistance to further traditional chemotherapy.

CSCs are believed to possess the following four characteristics:

1. Stemness—As used herein, stemness means the capacity to self-renew and differentiate into cancer cells (Gupta P B et al., Nat. Med. 2009; 15(9):1010-1012). While CSCs are only a minor portion of the total cancer cell population (Clarke M F, Biol. Blood Marrow Transplant. 2009; 11(2 suppl 2):14-16), they can give rise to heterogeneous lineages of cancer cells that make up the bulk of the tumor (see Gupta et al. 2009). In addition, CSCs possess the ability to mobilize to distinct sites while retaining their stemness properties and thus regrowth of the tumor at these sites (Jordan C T et al. N. Engl. J. Med. 2006; 355(12):1253-1261).

2. Aberrant signaling pathways—CSC stemness is associated with dysregulation of signaling pathways, which may contribute to their ability to regrow tumors and to migrate to distant sites. In normal stem cells, stemness signaling pathways are tightly controlled and genetically intact. In contrast, stemness signaling pathways in CSCs are dysregulated, allowing these cells to self-renew and differentiate into cancer cells (see Ajani et al. 2015). Dysregulation of stemness signaling pathways contributes to CSC resistance to chemotherapy and radiotherapy and to cancer recurrence and metastasis. Exemplary stemness signaling pathways involved in the induction and maintenance of stemness in CSCs include: JAK/STAT, Wnt/β-catenin, Hedgehog, Notch, and Nanog (Boman B M et al., J. Clin. Oncol. 2008; 26(17):2828-2838).

3. Resistance to traditional therapies—evidence suggests that CSCs possess resistance to conventional chemotherapy and radiation. While the detailed mechanism underlying such resistance is not well understood, the stemness pathways of CSCs (see Boman et al. 2008) together with the tumor microenvironment and aberrant regulation of signaling pathways (Borovski T. et al., Cancer Res. 2011; 71(3):634-639) may contribute to such resistance.

4. Ability to contribute to tumor recurrence and metastasis—although chemotherapy and radiation may kill most of the cells in a tumor, since CSCs are resistant to traditional therapies, the CSCs that are not eradicated may lead to regrowth or recurrence of the tumor either at the primary site or at distant sites (see Jordan et al. 2006). As mentioned above, CSCs may acquire the ability to mobilize to different sites and may maintain stemness at these sites through interactions with the microenvironment, allowing for metastatic tumor growth (see Boman et al. 2008).

The transcription factor Signal Transducer and Activator of Transcription 3 (referred to herein as Stat3) is a member of the Stat family, which are latent transcription factors activated in response to cytokines/growth factors to promote proliferation, survival, and other biological processes. Stat3 is an oncogene that can be activated by phosphorylation of a critical tyrosine residue mediated by growth factor receptor tyrosine kinases, including but not limited to, e.g., Janus kinases (JAKs), Src family kinases, EGFR, Abl, KDR, c-Met, and Her2. Yu, H. Stat3: Linking oncogenesis with tumor immune evasion in AACR 2008 Annual Meeting. 2008. San Diego, Calif. Upon tyrosine phosphorylation, the phosphorylated Stat3 (“pStat3”) forms homo-dimers and translocates to the nucleus, where it binds to specific DNA-response elements in the promoters of target genes, and induces gene expression. Pedranzini, L., et al. J. Clin. Invest., 2004. 114(5): p. 619-22.

In normal cells, Stat3 activation is transient and tightly regulated, lasting for example from 30 minutes to several hours. However, Stat3 is found to be aberrantly active in a wide variety of human cancers, including all the major carcinomas as well as some hematologic tumors. Persistently active Stat3 occurs in more than half of breast and lung cancers, colorectal cancers (CRC), ovarian cancers, hepatocellular carcinomas, multiple myelomas, etc., and in more than 95% of head/neck cancers. Stat3 plays multiple roles in cancer progression and is considered to be one of the major mechanisms for drug resistance to cancer cells. As a potent transcription regulator, Stat3 targets genes involved in cell cycle, cell survival, oncogenesis, tumor invasion, and metastasis, such as Bcl-xl, c-Myc, cyclin D1, Vegf, MMP-2, and survivin. Catlett-Falcone, R., et al. Immunity, 1999. 10(1): p. 105-15; Bromberg, J. F., et al. Cell, 1999. 98(3): p. 295-303; Kanda, N., et al. Oncogene, 2004. 23(28): p. 4921-29; Schlette, E. J., et al. J Clin Oncol, 2004. 22(9): p. 1682-88; Niu, G., et al. Oncogene, 2002. 21(13): p. 2000-08; Xie, T. X., et al. Oncogene, 2004. 23(20): p. 3550-60. It is also a key negative regulator of tumor immune surveillance and immune cell recruitment. Kortylewski, M., et al. Nat. Med., 2005. 11(12): p. 1314-21; Burdelya, L., et al. J. Immunol., 2005. 174(7): p. 3925-31; and Wang, T., et al. Nat. Med., 2004. 10(1): p. 48-54.

Abrogation of Stat3 signaling by using anti-sense oligonucleotides, siRNA, dominant-negative form of Stat3, and/or the targeted inhibition of tyrosine kinase activity causes cancer cell-growth arrest, apoptosis, and reduction of metastasis frequency both in vitro and/or in vivo. Pedranzini, L., et al. J Clin. Invest., 2004. 114(5): p. 619-22; Bromberg, J. F., et al. Cell, 1999. 98(3): p. 295-303; Darnell, J. E. Nat. Med., 2005. 11(6): p. 595-96; and Zhang, L., et al. Cancer Res, 2007. 67(12): p. 5859-64.

Furthermore, Stat 3 may play a role in the survival and self-renewal capacity of CSCs across a broad spectrum of cancers. Therefore, an agent with activity against CSCs may hold great promise for cancer patients (Boman, B. M., et al. J. Clin. Oncol. 2008. 26(17): p. 2795-99).

As discussed above, CSCs are a sub-population of cancer cells (found within solid tumors or hematological cancers) that possess characteristics normally associated with stem cells. These cells can grow faster after reduction of non-stem regular cancer cells by chemotherapy, which may be the mechanism for quick relapse after chemotherapies. In contrast to the bulk of cancer cells, which are non-tumorigenic, CSCs are tumorigenic (tumor-forming). In human acute myeloid leukemia, the frequency of these cells is less than 1 in 10,000. Bonnet, D. and J. E. Dick. Nat. Med., 1997. 3(7): p. 730-37. There is mounting evidence that such cells exist in almost all tumor types. However, as cancer cell lines are selected from a sub-population of cancer cells that are specifically adapted to growth in tissue culture, the biological and functional properties of these cell lines can change dramatically. Therefore, not all cancer cell lines contain CSCs.

CSCs have stem cell properties such as self-renewal and the ability to differentiate into multiple cell types. They persist in tumors as a distinct population and they give rise to the differentiated cells that form the bulk of the tumor mass and phenotypically characterize the disease. CSCs have been demonstrated to be fundamentally responsible for carcinogenesis, cancer metastasis, cancer recurrence, and relapse. CSCs are also called, for example, tumor initiating cells, cancer stem-like cells, stem-like cancer cells, highly tumorigenic cells, or super malignant cells.

CSCs are inherently resistant to conventional chemotherapies, which means they are left behind by conventional therapies that kill the bulk of tumor cells. As such, the existence of CSCs has several implications in terms of cancer treatment and therapy. These include, for example, disease identification, selective drug targets, prevention of cancer metastasis and recurrence, treatment of cancer refractory to chemotherapy and/or radiotherapy, treatment of cancers inherently resistant to chemotherapy or radiotherapy and development of new strategies in fighting cancer.

The efficacy of cancer treatments are, in the initial stages of testing, often measured by the amount of tumor mass they kill off. As CSCs form a very small proportion of the tumor cell population and have markedly different biologic characteristics than their differentiated progeny, the measurement of tumor mass may not select for drugs that act specifically on the stem cells. In fact, CSCs are radio-resistant and refractory to chemotherapeutic and targeted drugs. Normal somatic stem cells are naturally resistant to chemotherapeutic agents—they have various pumps (e.g., multidrug resistance protein pump) that efflux drugs, higher DNA repair capability, and have a slow rate of cell turnover (chemotherapeutic agents naturally target rapidly replicating cells). CSCs, being the mutated counterparts of normal stem cells, may also have similar functions that allow them to survive therapy. In other words, conventional chemotherapies kill differentiated (or differentiating) cells, which form the bulk of the tumor that is unable to generate new cells. A population of CSCs that gave rise to the tumor could remain untouched and cause a relapse of the disease. Furthermore, treatment with chemotherapeutic agents may only leave chemotherapy—resistant CSCs, so that the ensuing tumor will most likely also be resistant to chemotherapy. Cancer stem cells have also been demonstrated to be resistant to radiation therapy (XRT). Hambardzumyan, et al. Cancer Cell, 2006. 10(6): p. 454-56; and Baumann, M., et al. Nat. Rev. Cancer, 2008. 8(7): p. 545-54.

Since surviving CSCs can repopulate the tumor and cause relapse, anti-cancer therapies that include strategies against CSCs hold great promise. Jones R J et al., J Natl Cancer Inst. 2004; 96(8):583-585. By targeting CSC pathways, it may be possible to treat patients with aggressive, non-resectable tumors and refractory or recurrent cancers as well as prevent tumor metastasis and recurrence. Development of specific therapies targeting CSC pathways, therefore, may improve the survival and quality of life of cancer patients, especially those patients suffering from metastatic disease. Unlocking this untapped potential may involve the identification and validation of pathways that are selectively important for CSC self-renewal and survival. Though multiple pathways underlying tumorigenesis in cancer and in embryonic stem cells or adult stem cells have been elucidated in the past, pathways for cancer stem cell self-renewal and survival are still sought.

Methods for identification and isolation of CSCs have been reported. The methods used mainly exploit the ability of CSCs to efflux drugs or have been based on the expression of surface markers associated with cancer stem cells.

For example, since CSCs are resistant to many chemotherapeutic agents, it is not surprising that CSCs almost ubiquitously overexpress drug efflux pumps such as ABCG2 (BCRP-1), and other ATP binding cassette (ABC) superfamily members. Ho, M. M., et al. Cancer Res., 2007. 67(10): p. 4827-33; Wang, J., et al. Cancer Res., 2007. 67(8): p. 3716-24; Haraguchi, N., et al. Stem Cells, 2006. 24(3): p. 506-13; Doyle, L. A. and D. D. Ross. Oncogene, 2003. 22(47): p. 7340-58; Alvi, A. J., et al. Breast Cancer Res., 2003. 5(1): p. R1-R8; Frank, N. Y., et al. Cancer Res., 2005. 65(10): p. 4320-33; and Schatton, T., et al. Nature, 2008. 451(7176): p. 345-49. Accordingly, the side population (SP) technique, originally used to enrich hematopoetic and leukemic stem cells, was also employed to identify and isolate CSCs. Kondo, T., et al. Proc. Natl Acad. Sci. USA, 2004. 101(3): p. 781-86. This technique, first described by Goodell et al., takes advantage of differential ABC transporter-dependent efflux of fluorescent dyes such as Hoechst 33342 to define a cell population enriched in CSCs. Doyle, L. A. and D. D. Ross. Oncogene, 2003. 22(47): p. 7340-58; and Goodell, M. A., et al. J. Exp. Med., 1996. 183(4): p. 1797-806. Specifically, the SP is revealed by blocking drug efflux with verapamil, at which point the dyes can no longer be pumped out of the SP.

Efforts have also focused on finding specific markers that distinguish CSCs from the bulk of the tumor. Markers originally associated with normal adult stem cells have been found to also mark CSCs and co-segregate with the enhanced tumorigenicity of CSCs. Commonly expressed surface markers by the CSCs include CD44, CD133, and CD166. Al-Hajj, M., et al. Proc. Natl Acad. Sci. USA, 2003. 100(7): p. 3983-88; Collins, A. T., et al. Cancer Res., 2005. 65(23): p. 10946-51; Li, C., et al. Cancer Res., 2007. 67(3): p. 1030-37; Ma, S., et al. Gastroenterology, 2007. 132(7): p. 2542-56; Ricci-Vitiani, L., et al. Nature, 2007. 445(7123): p. 111-15; Singh, S. K., et al. Cancer Res., 2003. 63(18): p. 5821-28; and Bleau, A. M., et al., Neurosurg. Focus, 2008. 24(3-4): p. E28. Sorting tumor cells based primarily upon the differential expression of these surface marker(s) have accounted for the majority of the highly tumorigenic CSCs described to date. Therefore, these surface markers are validated for identification and isolation of CSCs from the cancer cell lines and from the bulk of tumor tissues.

By using aiRNA (asymmetric RNA duplexes), potent Stat3 selective silencing has been achieved in stemness-high cancer cells. This Stat3 silencing may lead to downregulation of cancer cell stemness, and/or inhibition of stemness-high cancer cell survival and self-renewal.

Furthermore, patients with higher expression levels of stemness genes have shown prolonged overall survival after treatment with a compound of formula (I) in clinical trials.

In some embodiments, the at least one compound of formula (I) is an inhibitor of CSC growth and survival. According to U.S. Pat. No. 8,877,803, the compound of formula (I) inhibits Stat3 pathway activity with a cellular IC₅₀ of ˜0.25 μM. The at least one compound of formula (I) may be synthesized according to U.S. Pat. No. 8,877,803, for example, Example 13. In some embodiments, the at least one compound of formula (I) is used in a method of treating cancers. According to PCT Patent Application No. PCT/US2014/033566, Example 6, the at least one compound of formula (I) was chosen to enter a clinical trial for patients with advanced cancers. The disclosures of U.S. Pat. No. 8,877,803 and PCT Patent Application No. PCT/US2014/033566 are incorporated herein by reference in their entireties.

We have surprisingly discovered that the at least one compound of formula (I) may re-sensitize a subject to at least one prior therapy even when the subject has developed, or started to develop, resistance or non-responsiveness to the at least one prior therapy. The at least one prior therapy can be chosen from anti-EGFR (epidermal growth factor receptor) therapy, cetuximab therapy, FOLFOX therapy, capecitabine therapy, and regorafenib therapy.

For instance, we have surprisingly discovered that a treatment combination of at least one compound of formula (I) and panitumumab results in anti-tumor activity in patients with certain types of cancer that have failed prior anti-EGFR therapy.

In some embodiments, disclosed herein are methods for resensitizing a subject to anti-EGFR therapy.

In some embodiments, disclosed herein are methods for simultaneously inhibiting, reducing, and/or diminishing (i) cancer stem cell survival and/or self-renewal and/or (ii) proliferation of heterogeneous cancer cells.

In some embodiments, these methods comprise administering to a subject in need thereof:

a therapeutically effective amount of at least one compound of formula (I) chosen from compounds having formula (I):

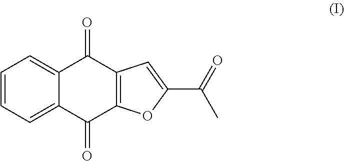 [SMILES: CC(=O)c1cc2c(o1)C(=O)c1ccccc1C2=O]

prodrugs, derivatives, pharmaceutically acceptable salts of any of the foregoing, and solvates of any of the; and

a therapeutically effective amount of at least one panitumumab compound chosen from panitumumab, pharmaceutically acceptable salts thereof, and solvates of any of the foregoing.

In some embodiments, provided herein are methods comprising administering to a subject in need thereof:

a therapeutically effective amount of at least one compound of formula (I) chosen from compounds having formula (I):

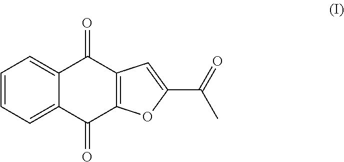 [SMILES: CC(=O)c1cc2c(o1)C(=O)c1ccccc1C2=O]

prodrugs, derivatives, pharmaceutically acceptable salts of any of the foregoing, and solvates of any of the; and

a therapeutically effective amount of at least one cetuximab compound chosen from cetuximab, pharmaceutically acceptable salts thereof, and solvates of any of the foregoing.

We have also surprisingly discovered that a treatment combination of at least one compound of formula (I) and FOLFOX results in anti-tumor activity in patients with certain types of cancer that have failed prior FOLFOX therapy.

In some embodiments, disclosed herein are methods for resensitizing a subject to FOLFOX therapy.

In some embodiments, disclosed herein are methods for simultaneously inhibiting, reducing, and/or diminishing (i) cancer stem cell survival and/or self-renewal and/or (ii) proliferation of heterogeneous cancer cells.

In some embodiments, these methods comprise administering to a subject in need thereof:

a therapeutically effective amount of at least one compound of formula (I) chosen from compounds having formula (I):

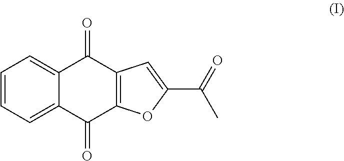 [SMILES: CC(=O)c1cc2c(o1)C(=O)c1ccccc1C2=O]

prodrugs, derivatives, pharmaceutically acceptable salts of any of the foregoing, and solvates of any of the; and

a therapeutically effective regimen of FOLFOX, and

optionally at least one angiogenesis inhibitor, for example, chosen from bevacizumab, its pharmaceutically acceptable salts, and solvates of any of the foregoing.

We also have also discovered that a treatment combination of at least one compound of formula (I) with capecitabine, with and without oxaliplatin, results in anti-tumor activity in patients with certain types of cancer.

In some embodiments, disclosed herein are methods for resensitizing a subject to capecitabine therapy.

In some embodiments, disclosed herein are methods for simultaneously inhibiting, reducing, and/or diminishing (i) cancer stem cell survival and/or self-renewal and/or (ii) proliferation of heterogeneous cancer cells.

In some embodiments, these methods comprise administering to a subject in need thereof:

a therapeutically effective amount of at least one compound chosen from compounds having formula (I):

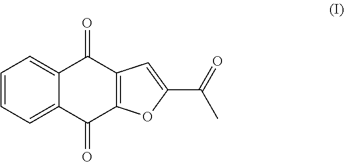 [SMILES: CC(=O)c1cc2c(o1)C(=O)c1ccccc1C2=O]

prodrugs, derivatives, pharmaceutically acceptable salts of any of the foregoing, and solvates of any of the foregoing, a therapeutically effective amount of capecitabine, and optionally at least one oxaliplatin compound chosen from oxaliplatin, pharmaceutically acceptable salts thereof, and solvates of any of the foregoing.

We also have also discovered that a treatment combination of at least one compound of formula (I) with regorafenib results in anti-tumor activity in patients with certain types of cancer.

In some embodiments, disclosed herein are methods for resensitizing a subject to regorafenib therapy.

In some embodiments, disclosed herein are methods for simultaneously inhibiting, reducing, and/or diminishing (i) cancer stem cell survival and/or self-renewal and/or (ii) proliferation of heterogeneous cancer cells.

In some embodiments, these methods comprise administering to a subject in need thereof:

a therapeutically effective amount of at least one compound chosen from compounds having formula (I):

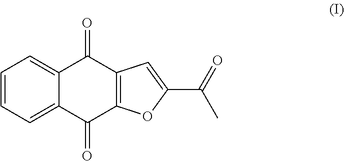 [SMILES: CC(=O)c1cc2c(o1)C(=O)c1ccccc1C2=O]

prodrugs, derivatives, pharmaceutically acceptable salts of any of the foregoing, and solvates of any of the foregoing, and a therapeutically effective amount of at least one regorafenib compound chosen from regorafenib, pharmaceutically acceptable salts thereof, and solvates of any of the foregoing.

In some embodiments, the cancer of any of the foregoing methods is chosen from colorectal cancer (e.g., K-Ras wild-type), esophageal cancer, esophageal adenocarcinoma, gastroesophageal junction cancer, gastroesophageal adenocarcinoma, chondrosarcoma, colorectal adenocarcinoma, rectal adenocarcinoma, colon adenocarcinoma, pancreatic adenocarcinoma, breast cancer, ovarian cancer, head and neck cancer, melanoma, gastric adenocarcinoma, gastroesophageal junction (GEJ) adenocarcinoma, adrenocorticoid carcinoma, cholangiocarcinoma, and hepatocellular carcinoma.

In some embodiments, a kit is disclosed that comprises: (1) at least one compound of formula (I); (2)(a) at least one panitumumab compound chosen from panitumumab, pharmaceutically acceptable salts thereof, and solvates of any of the foregoing; (b) at least one cetuximab compound chosen from cetuximab, pharmaceutically acceptable salts thereof, and solvates of any of the foregoing; (c) at least one leucovorin compound chosen from leucovorin, pharmaceutically acceptable salts thereof, and solvates of any of the foregoing, at least one 5-fluorouracil compound chosen from 5-fluorouracil, pharmaceutically acceptable salts thereof, and solvates of any of the foregoing, at least one oxaliplatin compound, or pharmaceutically acceptable salts thereof, and solvates of any of the foregoing, and optionally at least one angiogenesis inhibitor; (d) at least one capecitabine compound chosen from capecitabine, pharmaceutically acceptable salts thereof, and solvates of any of the foregoing and optionally at least one oxaliplatin compound chosen from oxaliplatin, pharmaceutically acceptable salts thereof, and solvates of any of the foregoing; or (e) at least one regorafenib compound chosen from regorafenib, pharmaceutically acceptable salts thereof, and solvates of any of the foregoing; and (3) instructions for administration and/or use.

Aspects and embodiments of the present disclosure are set forth or will be readily apparent from the following detailed description. It is to be understood that both the foregoing general description and the following detailed description are exemplary and explanatory only, and are not intended to be restrictive of the claims.

### BRIEF DESCRIPTION OF THE FIGURES

FIG. 1 shows the Stat3 pathway in cancer.

FIG. 2 shows the cancer stem cell specific and conventional cancer therapies.

FIG. 3 shows the initiation of relapse and metastases by cancer stem cells and cells with cancer stemness properties following treatment with conventional therapies.

FIG. 4(A), FIG. 4(B), FIG. 4(C), and FIG. 4(D) show the effect of treatment of 2-acetylnaphtho[2,3-b]furan-4,9-dione, Sunitinib, Gemcitabine, and Carboplatin, respectively, on β-Catenin, Nanog, Smo, and Sox2 levels in FaDu cell line.

FIG. 5 shows the effect of treatment of 2-acetylnaphtho[2,3-b]furan-4,9-dione on the protein levels of cancer stemness biomarkers p-Stat3 and β-catenin in human colon cancer xenograft Tumor (SW480) in nude mice.

FIG. 6 shows the effect of treatments on the protein levels of cancer stemness biomarkers p-Stat3 and β-catenin in a cancer xenograft tumor model.

FIG. 7 shows the greater than additive effect of the combination of 2-acetylnaphtho[2,3-b]furan-4,9-dione and 5-fluorouracil on cancer stem cells.

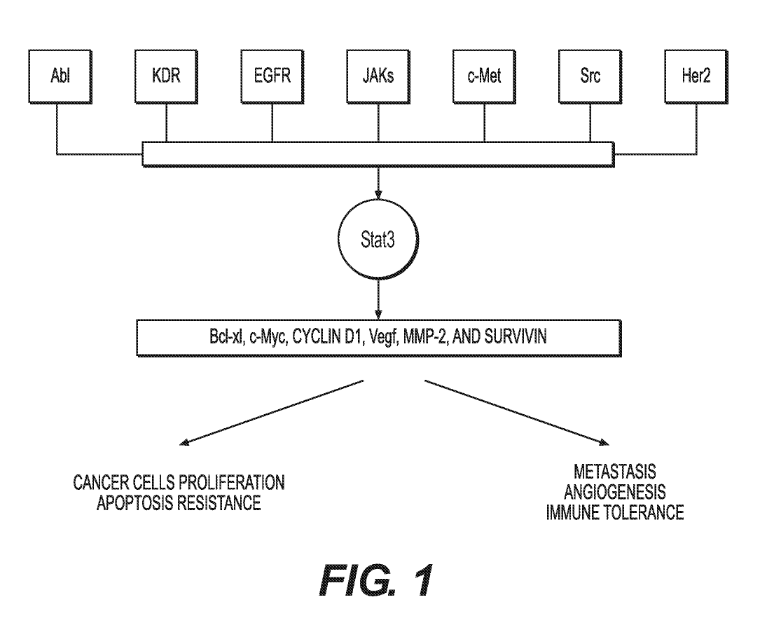

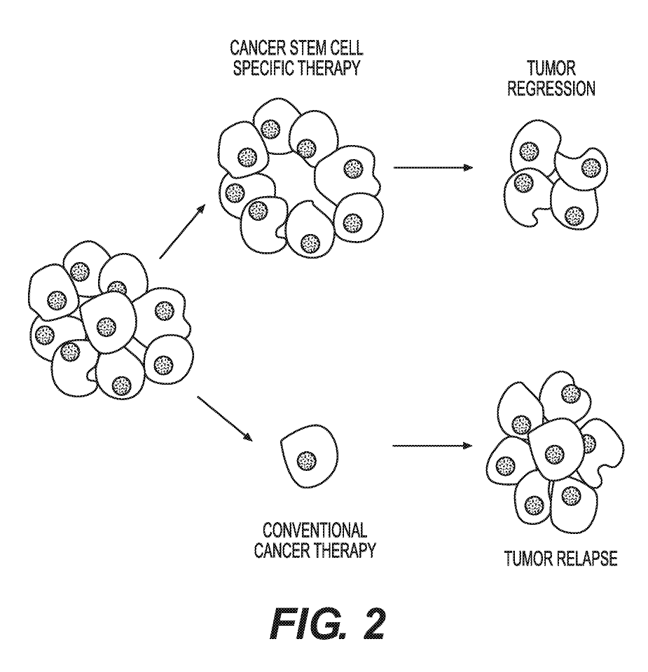

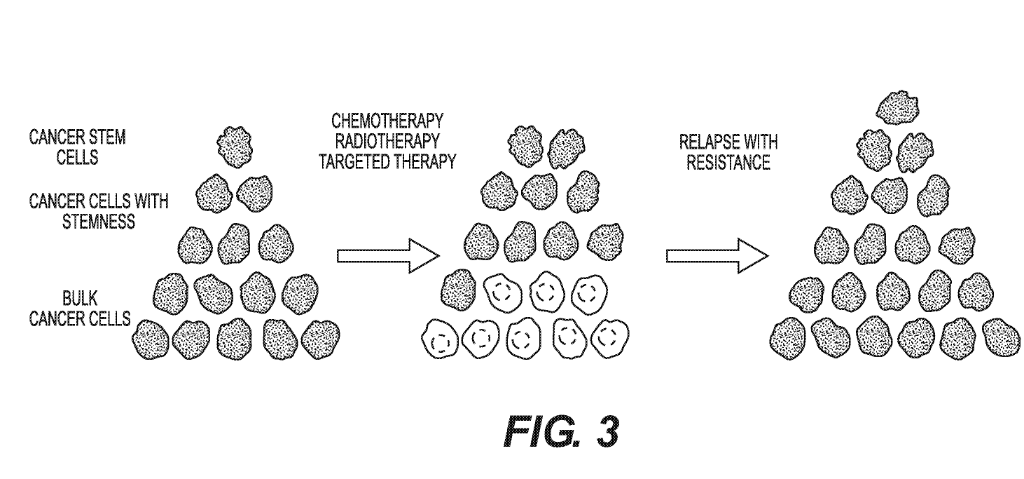

The following are definitions of terms used in the present specification. The initial definition provided for a group or term herein applies to that group or term throughout the present specification individually or as part of another group, unless otherwise indicated.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

The terms “administer,” “administering,” or “administration” are used herein in their broadest sense. These terms refer to any method of introducing to a subject a compound or pharmaceutical composition described herein and can include introducing the compound systemically, locally, or in situ to the subject. Thus, a compound of the present disclosure produced in a subject from a composition (whether or not includes the compound) is encompassed in these terms. When these terms are used in connection with the term “systemic” or “systemically,” they generally refer to in vivo systemic absorption or accumulation of the compound or composition in the blood stream followed by distribution throughout the entire body.

The term “subject” generally refers to an organism to which a compound or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to humans, mammals and non-mammals, such as non-human primates, mice, rabbits, sheep, dogs, cats, horses, cows, chickens, amphibians, and reptiles, which is to be the recipient of a compound or pharmaceutical composition described herein. Under some circumstances, the terms “subject” and “patient” are used interchangeably herein in reference to a human subject.

The terms “effective amount” and “therapeutically effective amount” refer to that amount of a compound or pharmaceutical composition described herein that is sufficient to effect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment,” “treating,” “ameliorating,” and “encouraging” may be used interchangeably herein. These terms refer to an approach for obtaining beneficial or desired results including, but not limited to, therapeutic benefit and/or prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject can still be afflicted with the underlying disorder. For prophylactic benefit, the pharmaceutical composition may be administered to a subject at risk of developing a particular disease, or to a subject reporting one or more of the physiological symptoms of a disease, even though a diagnosis of this disease may not have been made.

The term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, colon cancer, rectal cancer, cancer of the anal region, stomach cancer, gastric cancer, gastrointestinal cancer, gastric or gastroesophageal junction (GEJ) adenocarcinoma, adrenocorticoid carcinoma, hepatocellular carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, colorectal adenocarcinoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

In some embodiments, the cancer is colorectal cancer (e.g., K-Ras wild-type). In some embodiments, the cancer is colon adenocarcinoma. In some embodiments, the cancer is rectal adenocarcinoma. In some embodiments, the cancer is gastric adenocarcinoma. In some embodiments, the cancer is gastroesophageal junction (GEJ) adenocarcinoma. In some embodiments, the cancer is pancreatic adenocarcinoma. In some embodiments, the cancer is esophageal adenocarcinoma. In some embodiments, the cancer is cholangiocarcinoma. In some embodiments, the cancer is esophageal adenocarcinoma. In some embodiments, the cancer is hepatocellular carcinoma.

The terms “progress,” “progressed,” and “progression” refer to at least one of the following: (1) a response to prior therapy (e.g., chemotherapy) of progressive disease (PD); (2) the appearance of one or more new lesions after treatment with prior therapy (e.g., chemotherapy); and (3) at least a 5% (e.g., 10%, 20%) increase in the sum of diameters of target lesions, taking as a reference the smallest sum on study (this includes the baseline sum if that is the smallest on study).

As used herein, “re-sensitizing” means making subjects who were previously resistant, non-responsive, or somewhat responsive to a prior therapy (e.g., chemotherapy) regimen sensitive, responsive, or more responsive to that prior therapy (e.g., chemotherapy) regimen.

As used herein, the term “at least one compound of formula (I)” means at least one compound chosen from compounds having formula (I):

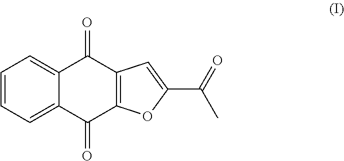 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, prodrugs and derivatives of compounds having formula (I) are Stat3 inhibitors. Non-limiting examples of prodrugs of compounds having formula (I) are the phosphoric ester and phosphoric diester described in U.S. pre-grant Publication No. 2012/0252763 as compound numbers 4011 and 4012 and also suitable compounds described in in U.S. Pat. No. 9,150,530. Non-limiting examples of derivatives of compounds having formula (I) include the derivatives disclosed in U.S. Pat. No. 8,877,803. The disclosures of U.S. pre-grant Publication No. 2012/0252763 and U.S. Pat. Nos. 9,150,530 and 8,877,803 are incorporated herein by reference.

Compounds having formula (I), shown below,

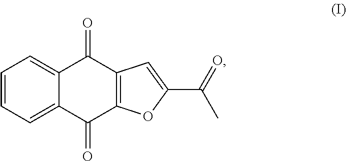 [SMILES: CC(=O)c1cc2c(o1)C(=O)c1ccccc1C2=O]

may also be known as 2-acetylnaphtho[2,3-b]furan-4,9-dione, napabucasin, or BBI608 and include tautomers thereof.

Suitable methods of preparing 2-acetylnaphtho[2,3-b]furan-4,9-dione, including its crystalline forms and additional cancer stemness inhibitors, are described in the co-owned PCT applications published as WO 2009/036099, WO 2009/036101, WO 2011/116398, WO 2011/116399, and WO 2014/169078; the contents of each application is incorporated herein by reference.

The term “salt(s),” as used herein, includes acidic and/or basic salts formed with inorganic and/or organic acids and bases. As used herein, the term “pharmaceutically acceptable salt” refers to those salts which are, within the scope of sound medical judgment, suitable for use in contact with the tissues of subjects without undue toxicity, irritation, allergic response and/or the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

Pharmaceutically acceptable salts may be formed with inorganic or organic acids. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Salts may be prepared in situ during the isolation and purification of the disclosed compound, or separately, such as by reacting the compound with a suitable base or acid, respectively. Non-limiting examples of pharmaceutically acceptable salts derived from bases include alkali metal, alkaline earth metal, ammonium and N⁺ (C₁₋₄alkyl)₄ salts. Non-limiting examples of suitable alkali or alkaline earth metal salts include sodium, lithium, potassium, calcium, magnesium, iron, zinc, copper, manganese, and aluminum salts. Further non-limiting examples of suitable pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Non-limiting examples of suitable organic bases from which salts may be derived include primary amines, secondary amines, tertiary amines, substituted amines including naturally occurring substituted amines, cyclic amines, and basic ion exchange resins, such as isopropylamine, trimethylamine, diethylamine, triethylamine, tripropylamine, and ethanolamine. In some embodiments, pharmaceutically acceptable base addition salts can be chosen from ammonium, potassium, sodium, calcium, and magnesium salts.

The term “solvate” represents an aggregate that comprises one or more molecules of a compound of the present disclosure with one or more molecules of a solvent or solvents. Solvates of the compounds of the present disclosure include, for example, hydrates.

The term “FOLFOX” as used herein refers to a combination therapy (e.g., chemotherapy) comprising at least one oxaliplatin compound chosen from oxaliplatin, pharmaceutically acceptable salts thereof, and solvates of any of the foregoing; at least one 5-fluorouracil (also known as 5-FU) compound chosen from 5-fluorouracil, pharmaceutically acceptable salts thereof, and solvates of any of the foregoing; and at least one folinic acid compound chosen from folinic acid (also known as leucovorin), levofolinate (the levo isoform of folinic acid), pharmaceutically acceptable salts of any of the foregoing, and solvates of any of the foregoing. The term “FOLFOX” as used herein is not intended to be limited to any particular amounts of or dosing regimens for those components. Rather, as used herein, “FOLFOX” includes all combinations of those components in any amounts and dosing regimens. As used herein, any recitation of the term “FOLFOX” may be replaced with a recitation of the individual components. For example, the term “FOLFOX” may be replaced with the phrase “at least one oxaliplatin compound chosen from oxaliplatin, pharmaceutically acceptable salts of oxaliplatin, solvates of oxaliplatin, and solvates of pharmaceutically acceptable salts of oxaliplatin; at least one 5-fluorouracil compound chosen from 5-fluorouracil, pharmaceutically acceptable salts of 5-fluorouracil, solvates of 5-fluorouracil, and solvates of pharmaceutically acceptable salts of 5-fluorouracil; and at least one folinic acid compound chosen from leucovorin, levofolinate, pharmaceutically acceptable salts of any of the foregoing, and solvates of any of the foregoing.”

A “therapeutically effective regimen” of FOLFOX, as used herein, means a therapeutically effective amount of the components of FOLFOX as defined herein administered according to a dosing regimen that is sufficient to effect the intended result including, but not limited to, disease treatment, as illustrated below. In some embodiments, a therapeutically effective regimen of FOLFOX comprises administering oxaliplatin together with leucovorin intravenously, followed by 5-FU intravenously. In some embodiments, a therapeutically effective regimen of FOLFOX comprises administering oxaliplatin in the amount of from about 50 mg/m² to about 200 mg/m² together with leucovorin in the amount of from about 200 mg/m² to about 600 mg/m² intravenously, followed by 5-FU in the amount of from about 1200 mg/m² to about 3600 mg/m² intravenously. In some embodiments, a therapeutically effective regimen of FOLFOX comprises administering oxaliplatin about 85 mg/m² together with leucovorin about 400 mg/m² intravenously, followed by 5-FU about 2400 mg/m². In some embodiments, a therapeutically effective regimen of FOLFOX comprises administering oxaliplatin 85 mg/m² together with leucovorin 400 mg/m² intravenously, followed by a 5-FU 400 mg/m² bolus and 5-FU 1200 mg/m²/day (total 2400 mg/m² over 46-48 hours) continuous intravenous infusion. In some embodiments, the above therapeutically effective regimen of FOLFOX is repeated every several days, for example, every 7 days, 14 days, or 21 days. In some embodiments, a therapeutically effective regimen of FOLFOX comprises: Day 1 oxaliplatin 85 mg/m² IV infusion and leucovorin 200 mg/m² IV infusion both given over 120 minutes at the same time in separate bags, followed by 5-FU 400 mg/m² IV bolus given over 2-4 minutes, followed by 5-FU 600 mg/m² IV infusion in 500 mL D5W as a 22-hour continuous infusion; Day 2 leucovorin 200 mg/m² IV infusion over 120 minutes, followed by 5-FU 400 mg/m² IV bolus given over 2-4 minutes, followed by 5-FU 600 mg/m² IV infusion as a 22-hour continuous infusion. In some embodiments, a therapeutically effective regimen of FOLFOX comprises: Day 1-2 oxaliplatin 100 mg/m² given as a 120 minute IV infusion, concurrent with leucovorin 400 mg/m² (or levoleucovorin 200 mg/m²) IV infusion, followed by 5-FU 400 mg/m² IV bolus, followed by 46-hour 5-FU infusion (2400 mg/m² for first two cycles, increased to 3000 mg/m² in case of no toxicity); Days 3-14: rest. In some embodiments FOLFOX is administered bi-weekly.

In some embodiments, panitumumab is administered weekly. In some embodiments, panitumumab at a dose of about 6 mg/kg is administered bi-weekly. In some embodiments, panitumumab at a dose of about 6 mg/kg is administered as an IV infusion over 60 minutes.

In some embodiments, cetuximab is administered weekly. In some embodiments, cetuximab at a dose of about 250 mg/m² is administered weekly. In some embodiments, cetuximab at a dose of about 250 mg/m² is administered as an IV infusion over 60 minutes. In some embodiments, one or more initial doses are administered. In some embodiments, an initial dose of 400 mg/m² is administered. In some embodiments, an initial dose of 400 mg/m² is administered as an IV infusion over 120 minutes.

In some embodiments, capecitabine is administered weekly. In some embodiments, capecitabine is administered orally in a divided dose, such as twice per day. In some embodiments, capecitabine is administered orally at a dose of 1000 mg/m² twice per day (BID) for 2 out of every 3 weeks.

In some embodiments, capecitabine is administered in combination with oxaliplatin. The term “CAPDX” as used herein refers to a combination therapy (e.g., chemotherapy) comprising at least one capecitabine compound chosen from capecitabine, pharmaceutically acceptable salts thereof, and solvates of any of the foregoing, and at least one oxaliplatin compound chosen from oxaliplatin, pharmaceutically acceptable salts thereof, and solvates of any of the foregoing. The term “CAPDX” as used herein is not intended to be limited to any particular amounts of or dosing regimens for these components. Rather, as used herein, “CAPDX” includes all combinations of these components in any amounts and dosing regimens. For example, the term “CAPDX” may be replaced with the phrase “at least one capecitabine compound chosen from capecitabine, pharmaceutically acceptable salts of capecitabine, solvates of capecitabine, and solvates of pharmaceutically acceptable salts of capecitabine; and at least one oxaliplatin compound chosen from oxaliplatin, pharmaceutically acceptable salts of oxaliplatin, solvates of oxaliplatin, and solvates of pharmaceutically acceptable salts of oxaliplatin.”

A “therapeutically effective regimen” of CAPDX, as used herein, means a therapeutically effective amount of the components of CAPDX as defined herein administered according to a dosing regimen that is sufficient to effect the intended result including, but not limited to, disease treatment, as illustrated below. In some embodiments, a therapeutically effective regimen of CAPDX operates in 3-week cycles, usually with 8 cycles in total. In some embodiments, capecitabine is taken orally twice daily for two weeks, while oxaliplatin is administered by IV on the first day of the cycle. In some embodiments, there is a one week rest period before the next cycle. In some embodiments, a therapeutically effective regimen of CAPDX comprises the administration of capecitabine 850 mg/m² BID orally and oxaliplatin 130 mg/m² IV. In some embodiments, a therapeutically effective regimen of CAPDX comprises the administration of capecitabine 850 mg/m² BID orally for 14 consecutive days and oxaliplatin 130 mg/m² IV every several days, for example, 21 days. In some embodiments, the therapeutically effective regimen is repeated every 21 days. If capecitabine is tolerated at the 850 mg/m² twice daily dose, dosage may be increased to 1000 mg/m² twice daily as tolerated after the first cycle.

In some embodiments, regorafenib is administered daily. In some embodiments, regorafenib is administered orally. In some embodiments, regorafenib is administered once per day. In some embodiments, regorafenib is administered orally at a dose of from about 100 mg to about 200 mg. In some embodiments, regorafenib is administered orally at a dose of 120 mg. In some embodiments, regorafenib is administered orally at a dose of 160 mg. In some embodiments, regorafenib is administered in a divided dose of four 40 mg tablets. In some embodiments, regorafenib is administered orally with a low-fat meal. In some embodiments, a therapeutically effective regimen of regorafenib includes four-week cycles, within each of which regorafenib is administered orally once daily for 7, 14, or 21 consecutive days. In some embodiments, a therapeutically effective regimen of regorafenib includes four-week cycles, within each of which regorafenib is administered orally once daily for 21 consecutive days.

In some embodiments, a compound of formula (I) may be administered with FOLFOX and at least one angiogenesis inhibitor. In some embodiments, the at least one angiogenesis inhibitor is chosen from bevacizumab and pharmaceutically acceptable salts thereof. In some embodiments, bevacizumab (e.g., about 5 mg/kg) is administered intravenously following infusion of folinic acid (or leucovorin calcium) and/or fluorouracil and/or oxaliplatin. In some embodiments, bevacizumab is administered bi-weekly.

The at least one compound disclosed herein may be in the form of a pharmaceutical composition. In some embodiments, the pharmaceutical compositions may comprise the at least one compound of formula (I) and at least one pharmaceutically acceptable carrier. In some embodiments, the pharmaceutical compositions may comprise one or more compounds and at least one pharmaceutically acceptable carrier, where the one or more compounds are capable of being converted into the at least one compound chosen from compounds of formula (I) and pharmaceutically acceptable salts and solvates thereof in a subject (i.e. a prodrug).

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

In some embodiments, the at least one compound may be administered in an amount ranging from about 160 to about 1500 mg. In some embodiments, the at least one compound may be administered in an amount ranging from about 160 to about 1000 mg. In some embodiments, the at least one compound may be administered in an amount ranging from about 300 mg to about 700 mg. In some embodiments, the at least one compound may be administered in an amount ranging from about 700 mg to about 1200 mg. In some embodiments, the at least one compound may be administered in an amount ranging from about 800 mg to about 1100 mg. In some embodiments, the at least one compound may be administered in an amount ranging from about 850 mg to about 1050 mg. In some embodiments, the at least one compound may be administered in an amount ranging from about 960 mg to about 1000 mg. In some embodiments, the total amount of the at least one compound is administered once daily. In some embodiments, the at least one compound is administered in a dose of about 480 mg daily. In some embodiments, the at least one compound is administered in administered in a dose of about 960 mg daily. In some embodiments, the at least one compound is administered in a dose of about 1000 mg daily. In some embodiments, the total amount of the at least one compound is administered in divided doses more than once daily, such as twice daily (BID) or more often. In some embodiments, the at least one compound may be administered in an amount ranging from about 80 to about 750 mg twice daily. In some embodiments, the at least one compound may be administered in an amount ranging from about 80 to about 500 mg twice daily. In some embodiments, the at least one compound is administered in a dose of about 240 mg twice daily. In some embodiments, the at least one compound is administered in administered in a dose of about 480 mg twice daily. In some embodiments, the at least one compound is administered in a dose of about 500 mg twice daily.

Pharmaceutical compositions disclosed herein that are suitable for oral administration may be in the form of capsules, cachets, pills, tablets, lozenges (using a flavored basis, usually sucrose and acacia or tragacanth), powders, granules, a solution in an aqueous or non-aqueous liquid, a suspension in an aqueous or non-aqueous liquid, an oil-in-water emulsion, a water-in-oil emulsion, an elixir, a syrup, pastilles (using an inert base, such as gelatin, glycerin, sucrose, and/or acacia) and/or mouthwashes, each containing a predetermined amount of the at least one compound of the present disclosure.

A pharmaceutical composition disclosed herein may be administered as a bolus, electuary, or paste.

Solid dosage forms for oral administration (capsules, tablets, pills, dragees, powders, granules and the like) may be mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type also may be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxypropyl-p-cyclodextrin, may be used to solubilize compounds.

The pharmaceutical compositions also may include adjuvants such as wetting agents, emulsifying and suspending agents, sweetening, flavoring, coloring, perfuming and preservative agents. Suspensions, in addition to the one or more compounds according to the disclosure, may contain suspending agents as, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions disclosed herein, for rectal or vaginal administration may be presented as a suppository, which may be prepared by mixing one or more compounds according to the disclosure, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the disclosure. Pharmaceutical compositions which are suitable for vaginal administration also may include pessaries, tampons, creams, gels, pastes, foams or spray formulations containing carriers that are known in the art to be appropriate.

Dosage forms for the topical or transdermal administration of a pharmaceutical composition or pharmaceutical tablet of the present disclosure may include powders, sprays, ointments, pastes, creams, lotions, gels, solutions, patches and inhalants. The pharmaceutical composition or pharmaceutical tablet may be mixed under sterile conditions with a pharmaceutically acceptable carrier, and with any preservatives, buffers, or propellants which may be required.

The ointments, pastes, creams and gels may contain, in addition to the pharmaceutical composition or pharmaceutical tablet of the present disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a pharmaceutical composition or a pharmaceutical tablet of the present disclosure, excipients such as lactose, talc, silicic acid, aluminum hydroxide, calcium silicates and polyamide powder, or mixtures of these substances. Additionally, sprays may contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of the present disclosure.

Compositions suitable for parenteral administration may comprise at least one more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In various embodiments, a composition described herein includes at least one compound chosen from compounds of formula (I) and pharmaceutically acceptable salts and solvates thereof and one or more surfactants. In some embodiments, the surfactant is sodium lauryl sulfate (SLS), sodium dodecyl sulfate (SDS), or one or more polyoxylglycerides. For example, the polyoxyglyceride can be lauroyl polyoxylglycerides (sometimes referred to as Gelucire) or linoleoyl polyoxylglycerides (sometimes referred to as Labrafil). Examples of such compositions are shown in PCT Patent Application No. PCT/US2014/033566, the contents of which are incorporated herein in its entirety.

As noted above, the methods disclosed herein may treat at least one disorder related to aberrant Stat3 pathway activity in a subject. Aberrant Stat3 pathway activity can be identified by expression of phosphorylated Stat3 (“pStat3”) or its surrogate upstream or downstream regulators.

The Stat3 pathway can be activated in response to cytokines, such as IL-6, or by a tyrosine kinase such as EGFR, JAKs, Abl, KDR, c-Met, Src, and Her2. The downstream effectors of Stat3 include but are not limited to Bcl-xl, c-Myc, cyclinD1, Vegf, MMP-2, and survivin. The Stat3 pathway has been found to be aberrantly active in a wide variety of cancers, as shown in Table 1. Persistently active Stat3 pathway may occur in more than half of breast and lung cancers, hepatocellular carcinomas, multiple myelomas and in more than 95% of head and neck cancers. Blocking the Stat3 pathway causes cancer cell-growth arrest, apoptosis, and reduction of metastasis frequency in vitro and/or in vivo.

TABLE 1
DISEASES

| ONCOLOGY | Solid tumors | Breast Cancer (Watson, C. J. and W. R. | |
|---|---|---|---|
| DISEASES | | Miller. Br. J. Cancer, 1995. 71(4): p. 840-44) | |
| | | Head and Neck Cancer (SCCHN) (Song, J. I. | |
| | | and J. R. Grandis. Oncogene, 2000. 19(21): | |
| | | p. 2489-95) | |
| | | Lung Cancer (Song, L., et al. Oncogene, | |
| | | 2003. 22(27): p. 4150-65) | |
| | | Ovarian Cancer (Savarese, T. M., et al. | |
| | | Cytokine, 2002. 17(6): p. 324-34) | |
| | | Pancreatic Cancer (Toyonaga, T., et al. | |
| | | Cancer Lett., 2003. 201(1): p. 107-16) | |
| | | Colorectal carcinoma (Corvinus, F. M., | |
| | | et al. Neoplasia, 2005. 7(6): p. 545-55) | |
| | | Prostate Cancer (Gao, B., et al. FEBS Lett., | |
| | | 2001. 488(3): p. 179-84) | |
| | | Renal Cell carcinoma (Buettner, R., et al. | |
| | | Clin. Cancer Res., 2002. 8(4): p. 945-54) | |
| | | Melanoma (Carson, W. E. Clin. Cancer Res., | |
| | | 1998. 4(9): p. 2219-28) | |
| | | Hepatocellular carcinomas (Darnell, J. E. | |
| | | Nat. Med., 2005. 11(6): p. 595-96) | |
| | | Cervical Cancer (Chen, C. L., et al. Br. J. | |
| | | Cancer, 2007. 96(4): p. 591-99) | |
| | | Endometrial Cancer (Chen, C. L., et al. Br. | |
| | | J. Cancer, 2007. 96(4): p. 591-99) | |
| | | Sarcomas (Lai, R., et al. J. Pathol., 2006. | |
| | | 208(5): p. 624-32; and) | |
| | | Brain Tumors (Punjabi, A. S., et al. J. Virol., | |
| | | 2007. 81(5): p. 2449-58) | |
| | | Gastric Cancers (Kanda, N., et al. Oncogene, | |
| | | 2004. 23(28): p. 4921-29) | |
| | Hematologic | Multiple Myeloma (Puthier, D., et al. Eur. J. | |
| | Tumors | Immunol., 1999. 29(12): p. 3945-50) | |
| | | Leukemia | HTLV-1-dependent Leukemia |
| | | | (Migone, T. S., et al. Science, |
| | | | 1995. 269(5220): p. 79-81) |
| | | | Chronic Myelogenous |
| | | | Leukemia (Buettner, R., et al. |
| | | | Clin. Cancer Res., 2002. 8(4): |
| | | | p. 945-54) |
| | | | Acute Myelogenous |
| | | | Leukemia (Spiekermann, K., |
| | | | et al. Eur. J. Haematol., 2001. |
| | | | 67(2): p. 63-71) |
| | | | Large Granular Lymphocyte |
| | | | Leukemia (Epling-Burnette, |
| | | | P. K., et al. J. Clin. Invest., |
| | | | 2001. 107(3): p. 351-62) |
| | | Lymphomas | EBV-related/Burkitt's |
| | | | (Weber-Nordt, R. M., et al. |
| | | | Blood, 1996. 88(3): p. 809-16) |
| | | | Mycosis Fungoides |
| | | | (Buettner, R., et al. Clin. |
| | | | Cancer Res., 2002. 8(4): p. |
| | | | 945-54) |
| | | | HSV Saimiri-dependent (T- |
| | | | cell) (Buettner, R., et al. Clin. |
| | | | Cancer Res., 2002. 8(4): p. |
| | | | 945-54) |
| | | | Cutaneous T-cell Lymphoma |
| | | | (Sommer, V. H., et al. |
| | | | Leukemia, 2004. 18(7): p. |
| | | | 1288-95) |
| | | | Hodgkin's Diseases |
| | | | (Buettner, R., et al. Clin. |
| | | | Cancer Res., 2002. 8(4): p. |
| | | | 945-54) |
| | | | Anaplastic Large-cell |
| | | | Lymphoma (Lai, R., et al. |
| | | | Am. J. Pathol., 2004. 164(6): |
| | | | p. 2251-58) |

In some embodiments, the at least one disorder may be chosen from cancers related to aberrant Stat3 pathway activity, such as colorectal cancer.

Recent studies have disclosed CSCs able to regenerate tumors. These CSCs are disclosed to be functionally linked with continued malignant growth, cancer metastasis, recurrence, and cancer drug resistance. CSCs and their differentiated progeny appear to have markedly different biologic characteristics. They persist in tumors as a distinct, but rare population. Conventional cancer drug screenings depend on measurement of the amount of tumor mass and, therefore, may not identify drugs that act specifically on the stem cells. In fact, CSCs have been disclosed to be resistant to standard chemotherapies and are enriched after standard chemotherapy treatments, which can result in refractory cancer and recurrence. CSCs have also been demonstrated to be resistant to radiotherapy. Baumann, M., et al. Nat. Rev. Cancer, 2008. 8(7): p. 545-54. The reported cancer types in which CSCs have been isolated include breast cancer, head cancer, neck cancer, lung cancer, ovarian cancer, pancreatic cancer, colorectal carcinoma, prostate cancer, melanoma, multiple myeloma, Kaposi sarcoma, Ewing's sarcoma, liver cancer, medulloblastoma, brain tumors, and leukemia. Stat3 has been identified as a CSC survival and self-renewal factor. Therefore, Stat3 inhibitors may kill CSCs and/or may inhibit CSC self-renewal. According to some embodiments, cancer stem cell or cancer stem cells refer to a minute population of cancer stem cells that have self-renewal capability and are tumorigenic.

Disclosed herein are methods of inhibiting, reducing, and/or diminishing CSC survival and/or self-renewal comprising administering a therapeutically effective amount of at least one pharmaceutical composition comprising at least one compound of formula (I) in combination with at least one additional anti-cancer therapy. Also disclosed herein are methods of inhibiting, reducing, and/or diminishing CSC survival and/or self-renewal comprising administering a therapeutically effective amount of at least one compound of formula (I) in combination with at least one additional anti-cancer therapy.

Also disclosed herein are methods of treating at least one cancer that is refractory to conventional chemotherapies and/or targeted therapies in a subject comprising administering a therapeutically effective amount of at least one compound of formula (I) in combination with at least one additional anti-cancer therapy. In various embodiments, the at least one compound is included in a pharmaceutical composition.

Disclosed herein are methods of treating recurrent cancer in a subject that has failed surgery, oncology therapy (e.g., chemotherapy), and/or radiation therapy, comprising administering a therapeutically effective amount of at least one compound of formula (I) in combination with at least one additional anti-cancer therapy. In various embodiments, the at least one compound is included in a pharmaceutical composition.

Also disclosed herein are methods of treating or preventing cancer metastasis in a subject, comprising administering a therapeutically effective amount of at least one compound of formula (I) in combination with at least one additional anti-cancer therapy. In various embodiments, the at least one compound is included in a pharmaceutical composition.

Disclosed herein are methods of treating cancer in a subject comprising administering a therapeutically effective amount of at least one compound of formula (I) in combination with at least one additional anti-cancer therapy.

In some embodiments, the at least one additional anti-cancer therapy is a therapeutically effective amount of at least one panitumumab compound chosen from panitumumab and/or pharmaceutically acceptable salts or solvates thereof. In some embodiments, the at least one additional anti-cancer therapy is a therapeutically effective amount of at least one cetuximab compound chosen from cetuximab and/or pharmaceutically acceptable salts or solvates thereof. In some embodiments, the at least one additional anti-cancer therapy is a therapeutically effective regimen FOLFOX, with or without a therapeutically effective amount of an angiogenesis inhibitor (e.g., bevacizumab). In some embodiments, the at least one additional anti-cancer therapy is a therapeutically effective amount of at least one capecitabine compound chosen from capecitabine, pharmaceutically acceptable salts thereof, and solvates of any of the foregoing, with or without a therapeutically effective amount of at least one oxaliplatin compound chosen from oxaliplatin, pharmaceutically acceptable salts thereof, and solvates of any of the foregoing. In some embodiments, the at least one additional anti-cancer therapy is a therapeutically effective amount of at least one regorafenib compound chosen from regorafenib, pharmaceutically acceptable salts thereof, and solvates of any of the foregoing.

In some embodiments, the cancer is chosen from colorectal cancer (e.g., K-Ras wild-type), esophageal cancer, esophageal adenocarcinoma, gastroesophageal junction cancer, gastroesophageal adenocarcinoma, chondrosarcoma, colorectal adenocarcinoma, rectal adenocarcinoma, colon adenocarcinoma, pancreatic adenocarcinoma, breast cancer, ovarian cancer, head and neck cancer, melanoma, gastric adenocarcinoma, gastroesophageal junction (GEJ) adenocarcinoma, adrenocorticoid carcinoma, cholangiocarcinoma, or hepatocellular carcinoma.

In some embodiments, the cancer may be advanced. In some embodiments, the cancer may be refractory. In some embodiments, the cancer may be recurrent. In some embodiments, the cancer may be metastatic. In some embodiments, the cancer may be associated with overexpression of Stat3. In some embodiments, the cancer may be associated with nuclear β-catenin localization.

### EXAMPLES

The methods disclosed herein comprise administering to a subject in need thereof a therapeutically effective amount of at least one compound of formula (I) and at least one additional anti-cancer therapy chosen from: (i) at least one panitumumab compound; (ii) at least one cetuximab compound; (iii) FOLFOX, with or without at least one angiogenesis inhibitor; (iv) at least one capecitabine compound with or without at least one oxaliplatin compound; and (v) at least one regorafenib compound.

### Example 1

The effects of 2-acetylnaphtho[2,3-b]furan-4,9-dione, a compound of formula (I), in combination with panitumumab in patients with metastatic colorectal cancer (mCRC) following progression on anti-EGFR therapy were studied in a phase Ib/II open label, multi-center study to assess the combination's safety and preliminary anti-cancer activity.

Following clearance of the dose-limiting toxicity (DLT) period in 6 patients, 18 more patients were enrolled. All of the 24 patients enrolled as of April 2015 were pre-treated with >2 lines of therapy and 12/24 (50%) with >3 lines of therapy.

Patients with advanced K-Ras wild-type mCRC received oral administration of 2-acetylnaphtho[2,3-b]furan-4,9-dione twice daily together with panitumumab. Specifically, 2-acetylnaphtho[2,3-b]furan-4,9-dione was administered at a dose of 480 mg or 500 mg BID in combination with panitumumab at 6 mg/kg bi-weekly until progression of disease, unacceptable toxicity, or other discontinuation criterion was met.

The combination of 2-acetylnaphtho[2,3-b]furan-4,9-dione together with panitumumab demonstrated anti-cancer activity. For example, disease control (stable disease (SD)+objective partial response (PR)) was observed in 4 of 9 (44.4%) anti-EGFR naïve patients. Of those patients, 2 of 9 (22%) had PR (35.5% and 33.3% regressions), and 2 had SD.

Surprisingly, disease control (only SD) was observed in 8 of 15 (53.3%) patients who had failed anti-EGFR (cetuximab) therapy, 2 of which had SD with regression (12.9% and 6.8%). Without being limited to any particular theory, the presence of 2-acetylnaphtho[2,3-b]furan-4,9-dione appeared to re-sensitize the patients to the panitumumab treatment even when these patients had developed or started to develop resistance to the panitumumab treatment.

The median progression-free survival (mPFS) was 9 weeks and 16.4 weeks in anti-EGFR naïve and previously exposed patients, respectively.

The combination of 2-acetylnaphtho[2,3-b]furan-4,9-dione plus bi-weekly panitumumab was well tolerated. A maximum tolerated dose (MTD) was not determined and 2-acetylnaphtho[2,3-b]furan-4,9-dione could be given in combination with full dose panitumumab. This therapy also exhibited a safety profile similar to that of each regimen as monotherapy. The most common adverse events included grade 1-2 diarrhea, abdominal cramps, nausea and vomiting. Grade 3 hypokalemia and dehydration occurred in 2 patients. No significant pharmacokinetic interactions were observed.

This phase Ib/II study demonstrated that 2-acetylnaphtho[2,3-b]furan-4,9-dione and bi-weekly panitumumab can be safely combined at full dose. The response rate in anti-EGFR naïve patients was notably greater than that of anti-EGFR monotherapy reported historically. Moreover, encouraging preliminary anti-cancer activity was observed in K-Ras wild-type mCRC patients regardless of prior anti-EGFR exposure.

This study was continued, and the updated results in Tables 2-4 showed similar observations for 480 mg BID 2-acetylnaphtho[2,3-b]furan-4,9-dione:

TABLE 2
Response rates (Evaluable)

| Combination | Patients | DCR | ORR |
|---|---|---|---|
| Panitumumab + | N = 47 | 51.1% | 6.4% |
| 2-acetylnaphtho[2,3-b]furan- | | | |
| 4,9-dione | | | |

TABLE 3

| | Historical DCR (in Evaluable |
|---|---|
| Single Agent | patients with mCRC) |
| Panitumumab | 30-40% (K-Ras WT) |

TABLE 4
Response rates (ITT)

| | Patients | DCR | ORR |
|---|---|---|---|
| Panitumumab + | N = 71 | 33.8% | 4.2% |
| 2-acetylnaphtho[2,3-b]furan- | | | |
| 4,9-dione | | | |

TABLE 5

| | Evaluable | | |
|---|---|---|---|
| | patients | DCR (SD + PR) | Comments |
| Anti-EGFR naïve | N = 18 | 55.5% (10/18) | 7 SD + 3 PR |
| Anti-EGFR exposed | N = 35 | 48.6% (17/35) | 16 SD + 1 PR |
| including | | | |
| anti-EGFR exposed | N = 15 | 53.3% (8/15) | 8 SD |
| and progressed | | | |

In the evaluable patients (N=47) receiving a combination of 2-acetylnaphtho[2,3-b]furan-4,9-dione and panitumumab, the DCR was 51.1% and the objective response rate (ORR) was 6.4% (see Table 2). In contrast, the historical DCR for panitumumab was 30-40% in K-Ras wild-type mCRC patients (see Table 3).

Similarly, in intent to treat population (ITT), response rates for 2-acetylnaphtho[2,3-b]furan-4,9-dione in combination with panitumumab were examined. The combination of 2-acetylnaphtho[2,3-b]furan-4,9-dione and panitumumab resulted in a DCR of 33.8% and an objective response rate of 4.2% (see Table 4).

As shown in Table 5, the combination again proved efficacious in anti-EGFR exposed patients who had progressed on prior anti-EGFR therapy. For example, the disease control rate (DCR) (stable disease (SD)+objective partial response (PR)) was observed in 17 out of 35 (48.6%) of such patients.

In a separate arm, patients with advanced K-Ras wild-type mCRC received oral administration of 2-acetylnaphtho[2,3-b]furan-4,9-dione twice daily together with cetuximab. Specifically, 2-acetylnaphtho[2,3-b]furan-4,9-dione was administered at a dose of 480 mg or 500 mg BID in combination with cetuximab administered IV at 400 mg/m² intravenous infusion over 120 minutes as the initial dose, then weekly at 250 mg/m² over 60-minutes at subsequent cycles or until progression of disease, unacceptable toxicity, or other discontinuation criterion was met.

The combination of 2-acetylnaphtho[2,3-b]furan-4,9-dione together with cetuximab demonstrated anti-cancer activity. For example, the disease control rate (DCR) and the objective response rate (ORR) in the evaluable patients were observed at 44.4% and 11.1%, respectively; and the disease control rate (DCR) and the objective response rate (ORR) in the intent to treat population (ITT) were observed at 31.3% and 6.2%, respectively. In contrast, the historical DCR for cetuximab is 30-40%.

### Example 2

The effects of 2-acetylnaphtho[2,3-b]furan-4,9-dione in combination with capecitabine in patients with advanced mCRC was investigated.

All patients received oral administration of 2-acetylnaphtho[2,3-b]furan-4,9-dione twice daily together with capecitabine. In particular, 2-acetylnaphtho[2,3-b]furan-4,9-dione was administered at a dose of 480 mg BID in combination with capecitabine until progression of disease, unacceptable toxicity, or other discontinuation criterion was met. Capecitabine was administered orally at 1000 mg/m² bid daily on days 8-21 every three weeks or until progression of disease, unacceptable toxicity, or other discontinuation criterion was met.

The combination of 2-acetylnaphtho[2,3-b]furan-4,9-dione and capecitabine demonstrated a DCR of 50.0% and an ORR of 5.8% in the evaluable patients (N=52). In contrast, the historic DCR for capecitabine was 15% (compare Tables 6 and 7).

TABLE 6
Response rates (Evaluable)

| Combination | Patients | DCR | ORR |
|---|---|---|---|
| Capecitabine + | N = 52 | 50.0% | 5.8% |
| 2-acetylnaphtho[2,3-b]furan- | | | |
| 4,9-dione | | | |

TABLE 7

| | Historical DCR (in Evaluable |
|---|---|
| Single Agent | patients with mCRC) |
| Capecitabine | 15% |

Similarly, in intent to treat population (ITT), response rates for 2-acetylnaphtho[2,3-b]furan-4,9-dione in combination with capecitabine were examined (see Table 8). The combination of 2-acetylnaphtho[2,3-b]furan-4,9-dione and capecitabine demonstrated a DCR of 33.8% and an ORR of 3.9%.

TABLE 8
Response rates (ITT)

| | Patients | DCR | ORR |
|---|---|---|---|
| Capecitabine + | N = 77 | 33.8% | 3.9% |
| 2-acetylnaphtho[2,3-b]furan- | | | |
| 4,9-dione | | | |

### Example 3

The safety, tolerability, and preliminary anti-tumor activity of 2-acetylnaphtho[2,3-b]furan-4,9-dione in combination with capecitabine as part of a CAPDX regimen was investigated in adult patients with colon adenocarcinoma.

Eight patients aged 42-73 who were pretreated with 2-4 prior lines of therapy received 2-acetylnaphtho[2,3-b]furan-4,9-dione with CAPDX. Patients received continuous oral administration of 2-acetylnaphtho[2,3-b]furan-4,9-dione twice daily. For instance, 2-acetylnaphtho[2,3-b]furan-4,9-dione was administered at a dose of 240 mg BID or 480 mg BID in combination with CAPDX until progression of disease, unacceptable toxicity, or other discontinuation criterion was met. CAPDX was administered orally (capecitabine) and IV (oxaliplatin). Capecitabine 850 mg/m² was administered orally twice-daily for 14 consecutive days and repeated every 21 days or until progression of disease, unacceptable toxicity, or other discontinuation criterion was met. Oxaliplatin 130 mg/m² was administered IV and repeated every 21 days thereafter. If capecitabine was tolerated at the 850 mg/m² twice daily dose, dosage was increased to 1000 mg/m² twice daily as tolerated after the first cycle.

Objective tumor response was assessed every 8 weeks using Response Evaluation Criteria in Solid Tumors (RECIST 1.1).

This study demonstrated that 2-acetylnaphtho[2,3-b]furan-4,9-dione dosed at 240 mg BID or 480 mg BID can be safely combined with CAPDX. For example, as shown in Table 9, a number of patients had stable disease (SD).

### Example 4

The effects of 2-acetylnaphtho[2,3-b]furan-4,9-dione in combination with capecitabine as part of a CAPDX regimen was assessed in adult patients with gastric and gastroesophageal junction (GEJ) adenocarcinoma.

Two patients aged 55 and 71 who were pretreated with 0 or 4 lines of prior treatment received oral administration of 2-acetylnaphtho[2,3-b]furan-4,9-dione twice daily together with CAPDX. For example, 2-acetylnaphtho[2,3-b]furan-4,9-dione was administered at a dose of 240 mg BID in combination with CAPDX until progression of disease, unacceptable toxicity, or other discontinuation criterion was met. CAPDX was administered orally (capecitabine) and IV (oxaliplatin). Capecitabine 850 mg/m² was administered orally twice-daily for 14 consecutive days and repeated every 21 days or until progression of disease, unacceptable toxicity, or other discontinuation criterion was met. Oxaliplatin 130 mg/m² was administered IV and repeated every 21 days thereafter. If capecitabine was tolerated at the 850 mg/m² twice daily dose, dosage was increased to 1000 mg/m² twice daily as tolerated after the first cycle.

Anticancer activity was observed with this regimen. For example, one patient had stable disease (SD) (see Table 9).

TABLE 9
(“BBI608” = 2-acetylnaphtho[2,3-b]furan-4,9-dione)

| | | | | | | Prior | |
|---|---|---|---|---|---|---|---|
| | BBI608 | | | Chemo | Prior | # of | Best |
| Patient | (mg) | | Diagnosis | Backbone | Response | Lines | Response |
| 0068 | 240 | bid | Gastric adenocarcinoma | CAPOX | N/A | 4 | SD |
| 0207 | 240 | bid | Gastric adenocarcinoma | CAPOX | N/A | 0 | Pending |

### Example 5

The effects of 2-acetylnaphtho[2,3-b]furan-4,9-dione in combination with capecitabine as part of a CAPDX regimen was assessed in adult patients with pancreatic adenocarcinoma.

Nine patients aged 55-78 who were pretreated with 1-3 lines of prior treatment received oral administration of 2-acetylnaphtho[2,3-b]furan-4,9-dione twice daily together with CAPDX. For example, 2-acetylnaphtho[2,3-b]furan-4,9-dione was administered at a dose of 240 mg BID or 480 mg BID in combination with CAPDX until progression of disease, unacceptable toxicity, or other discontinuation criterion was met. CAPDX was administered orally (capecitabine) and IV (oxaliplatin). Capecitabine 850 mg/m² was administered orally twice-daily for 14 consecutive days and repeated every 21 days or until progression of disease, unacceptable toxicity, or other discontinuation criterion was met. Oxaliplatin was administered IV and repeated every 21 days thereafter. If capecitabine was tolerated at the 850 mg/m² twice daily dose, dosage was increased to 1000 mg/m² twice daily as tolerated after the first cycle.

Anticancer activity was observed with this regimen. For example, three patients had stable disease (SD).

### Example 6

The effects of 2-acetylnaphtho[2,3-b]furan-4,9-dione in combination with capecitabine as part of a CAPDX regimen was assessed in adult patients with esophageal adenocarcinoma.

Four patients aged 58-82 who were pretreated with 0-3 lines of prior treatment received oral administration of 2-acetylnaphtho[2,3-b]furan-4,9-dione twice daily together with CAPDX. For example, 2-acetylnaphtho[2,3-b]furan-4,9-dione was administered at a dose of 240 mg BID or 480 mg BID in combination with CAPDX until progression of disease, unacceptable toxicity, or other discontinuation criterion was met. CAPDX was administered orally (capecitabine) and IV (oxaliplatin). Capecitabine 850 mg/m² was administered orally twice-daily for 14 consecutive days and repeated every 21 days or until progression of disease, unacceptable toxicity, or other discontinuation criterion was met. Oxaliplatin 130 mg/m² was administered IV and repeated every 21 days thereafter. If capecitabine was tolerated at the 850 mg/m² twice daily dose, dosage was increased to 1000 mg/m² twice daily as tolerated after the first cycle.

Anticancer activity was observed with this regimen. For example, one patient had stable disease (SD) (see Table 10)

TABLE 10
(“BBI608” = 2-acetylnaphtho[2,3-b]furan-4,9-dione)

| | | | | | Prior | |
|---|---|---|---|---|---|---|
| | BBI608 | | | Chemo | # of | Best |
| Patient | (mg) | | Diagnosis | Backbone | Lines | Response |
| 0012 | 240 | bid | Esophageal | CAPOX | 0 | PR; 50% |
| | | | adenocarcinoma | | | regression |
| 0145 | 240 | bid | Esophageal | CAPOX | 1 | NE |
| | | | adenocarcinoma | | | |
| 0250 | 240 | bid | Esophageal | CAPOX | 3 | pending |
| | | | adenocarcinoma | | | |
| 0021 | 480 | bid | Esophageal | CAPOX | 0 | SD; 24% |
| | | | adenocarcinoma | | | regression |

### Example 7

The effects of 2-acetylnaphtho[2,3-b]furan-4,9-dione in combination with capecitabine as part of a CAPDX regimen was assessed in adult patients with cholangiocarcinoma.

Eight patients aged 51-77 who were pretreated with 1-3 lines of prior treatment received oral administration of 2-acetylnaphtho[2,3-b]furan-4,9-dione twice daily together with CAPDX. For example, 2-acetylnaphtho[2,3-b]furan-4,9-dione was administered at a dose of 240 mg BID or 480 mg BID in combination with CAPDX until progression of disease, unacceptable toxicity, or other discontinuation criterion was met. CAPDX was administered orally (capecitabine) and IV (oxaliplatin). Capecitabine 850 mg/m² was administered orally twice-daily for 14 consecutive days and repeated every 21 days or until progression of disease, unacceptable toxicity, or other discontinuation criterion was met. Oxaliplatin 130 mg/m² was administered IV and repeated every 21 days thereafter. If capecitabine was tolerated at the 850 mg/m² twice daily dose, dosage was increased to 1000 mg/m² twice daily as tolerated after the first cycle.

Anticancer activity was observed with this regimen. For example, two patients had a partial response (PR) with tumor regression and one patient had stable disease (SD).

### Example 8

The effects of 2-acetylnaphtho[2,3-b]furan-4,9-dione in combination with capecitabine as part of a CAPDX regimen was assessed in adult patients with hepatocelular carcinoma.

Four patients aged 21-69 who were pretreated with 0-3 lines of prior treatment received oral administration of 2-acetylnaphtho[2,3-b]furan-4,9-dione twice daily together with CAPDX. For example, 2-acetylnaphtho[2,3-b]furan-4,9-dione was administered at a dose of 240 mg BID or 480 mg BID in combination with CAPDX until progression of disease, unacceptable toxicity, or other discontinuation criterion was met. CAPDX was administered orally (capecitabine) and IV (oxaliplatin). Capecitabine 850 mg/m² was administered orally twice-daily for 14 consecutive days and repeated every 21 days or until progression of disease, unacceptable toxicity, or other discontinuation criterion was met. Oxaliplatin 130 mg/m² was administered IV and repeated every 21 days thereafter. If capecitabine was tolerated at the 850 mg/m² twice daily dose, dosage was increased to 1000 mg/m² twice daily as tolerated after the first cycle.

Anticancer activity was observed with this regimen. For example, one patient had a partial response (PR).

### Example 9

The effects of 2-acetylnaphtho[2,3-b]furan-4,9-dione in combination with regorafenib in patients with colon adenocarcinoma and rectal adenocarcinoma were investigated.

The patients in this study were aged 36-82 and were pretreated with at least 2 prior lines of therapy.

These patients received oral administration of 2-acetylnaphtho[2,3-b]furan-4,9-dione twice daily together with regorafenib. For example, 2-acetylnaphtho[2,3-b]furan-4,9-dione was administered at a dose of 240 mg BID or 480 BID in combination with regorafenib at an oral dose of 120 mg once daily with a low-fat meal and be continued for 21 consecutive days of every 28 days thereafter or until progression of disease, unacceptable toxicity, or other discontinuation criterion was met. If regorafenib was tolerated in the first cycle, dosage was increased to 160 mg once daily as tolerated after the first cycle.

As shown in Table 15, disease control (stable disease (SD)) was observed in a number of patients. Thus, 2-acetylnaphtho[2,3-b]furan-4,9-dione twice daily together with regorafenib resulted in anti-cancer activity.

### Example 10

The safety, tolerability, and preliminary anti-tumor activity of 2-acetylnaphtho[2,3-b]furan-4,9-dione in combination with FOLFOX, with and without bevacizumab, were investigated in adult patients with colon adenocarcinoma or rectal adenocarcinoma.

### Example 10a

Patients aged 40-77 who were pretreated with 0-5 prior lines of therapy received 2-acetylnaphtho[2,3-b]furan-4,9-dione in combination with FOLFOX without bevacizumab. Patients received continuous oral administration of 2-acetylnaphtho[2,3-b]furan-4,9-dione twice daily. For instance, 2-acetylnaphtho[2,3-b]furan-4,9-dione was administered at a dose of 240 mg BID or 480 mg BID in combination with FOLFOX until progression of disease, unacceptable toxicity, or other discontinuation criterion was met. Oxaliplatin 85 mg/m² together with leucovorin 400 mg/m² were administered intravenously. 5-FU 400 mg/m² bolus was administered intravenously immediately following oxaliplatin/leucovorin infusion, followed by 5-FU 1200 mg/m²/day (total 2400 mg/m² over 46-48 hours) continuous intravenous infusion. This regimen was repeated every 14 days thereafter or until progression of disease, unacceptable toxicity, or other discontinuation criterion was met.

Objective tumor response was assessed every 8 weeks using Response Evaluation Criteria In Solid Tumors (RECIST 1.1).

This study demonstrated that 2-acetylnaphtho[2,3-b]furan-4,9-dione dosed at 240 mg BID or 480 mg BID can be safely combined with FOLFOX. Surprisingly, as shown in Table 16, anti-cancer activity was observed in patients who had failed prior FOLFOX chemotherapy. Without being limited to any particular theory, the presence of 2-acetylnaphtho[2,3-b]furan-4,9-dione appeared to re-sensitize the patients to the FOLFOX regimen even when these patients had developed or started to develop resistance to FOLFOX treatment.

### Example 10b

Patients aged 24-79 who were pretreated with 0-6 prior lines of therapy received 2-acetylnaphtho[2,3-b]furan-4,9-dione in combination with FOLFOX and bevacizumab. Patients received continuous oral administration of 2-acetylnaphtho[2,3-b]furan-4,9-dione twice daily at a dose of 240 mg BID or 480 mg BID. Oxaliplatin 85 mg/m² together with leucovorin 400 mg/m² were administered intravenously. 5-FU 400 mg/m² bolus was administered intravenously immediately following oxaliplatin/leucovorin infusion, followed by 5-FU 1200 mg/m²/day (total 2400 mg/m² over 46-48 hours) continuous intravenous infusion. This regimen was repeated every 14 days thereafter or until progression of disease, unacceptable toxicity, or other discontinuation criterion was met. Bevacizumab 5 mg/kg was administered intravenously following oxaliplatin/leucovorin infusion until progression of disease, unacceptable toxicity, or other discontinuation criterion was met.

Objective tumor response was assessed every 8 weeks using Response Evaluation Criteria In Solid Tumors (RECIST 1.1).

This study demonstrated that 2-acetylnaphtho[2,3-b]furan-4,9-dione dosed at 240 mg BID or 480 mg BID can be safely combined with FOLFOX and bevacizumab. For example, a number of patients had partial response (PR) or stable disease (SD).

### Example 11

The effects of 2-acetylnaphtho[2,3-b]furan-4,9-dione in combination with FOLFOX were assessed in adult patients with gastric and gastroesophageal junction_(GEJ) adenocarcinoma.

Patients aged 45-78 who were pretreated with 0-4 lines of prior treatment received oral administration of 2-acetylnaphtho[2,3-b]furan-4,9-dione twice daily together with FOLFOX. For example, 2-acetylnaphtho[2,3-b]furan-4,9-dione was administered at a dose of 240 mg BID or 480 mg BID in combination with FOLFOX. Oxaliplatin 85 mg/m² together with leucovorin 400 mg/m² were administered intravenously. 5-FU 400 mg/m² bolus was administered intravenously immediately following oxaliplatin/leucovorin infusion, followed by 5-FU 1200 mg/m²/day (total 2400 mg/m² over 46-48 hours) continuous intravenous infusion. This regimen was repeated every 14 days thereafter or until progression of disease, unacceptable toxicity, or other discontinuation criterion was met.

Anti-cancer activity was observed with this regimen. For example, a number of patients had partial response (PR) or stable disease (SD).

### Example 12

The effects of 2-acetylnaphtho[2,3-b]furan-4,9-dione in combination with FOLFOX were investigated in adult patients with pancreatic adenocarcinoma in a clinical study.

In this study, patients aged 52-79 received oral administration of 2-acetylnaphtho[2,3-b]furan-4,9-dione twice daily together with FOLFOX. Specifically, 2-acetylnaphtho[2,3-b]furan-4,9-dione was administered at a dose of 240 mg BID in combination with FOLFOX. Oxaliplatin 85 mg/m² together with leucovorin 400 mg/m² were administered intravenously. 5-FU 400 mg/m² bolus was administered intravenously immediately following oxaliplatin/leucovorin infusion, followed by 5-FU 1200 mg/m²/day (total 2400 mg/m² over 46-48 hours) continuous intravenous infusion. This regimen was repeated every 14 days thereafter or until progression of disease, unacceptable toxicity, or other discontinuation criterion was met.

The combination of 2-acetylnaphtho[2,3-b]furan-4,9-dione twice daily together with FOLFOX resulted in partial response (PR) or stable disease (SD) in a number of patients, establishing anti-cancer activity for this combination.

### Example 13

The effects of 2-acetylnaphtho[2,3-b]furan-4,9-dione in combination with FOLFOX were assessed in adult patients with esophageal adenocarcinoma.

Patients aged 64 and 71 who were pretreated with 1 line of prior treatment received oral administration of 2-acetylnaphtho[2,3-b]furan-4,9-dione twice daily together with FOLFOX. For example, 2-acetylnaphtho[2,3-b]furan-4,9-dione was administered at a dose of 240 mg BID in combination with FOLFOX. Oxaliplatin 85 mg/m² together with leucovorin 400 mg/m² were administered intravenously. 5-FU 400 mg/m² bolus was administered intravenously immediately following oxaliplatin/leucovorin infusion, followed by 5-FU 1200 mg/m²/day (total 2400 mg/m² over 46-48 hours) continuous intravenous infusion. This regimen was repeated every 14 days thereafter or until progression of disease, unacceptable toxicity, or other discontinuation criterion was met.

Anti-cancer activity was observed with this regimen. For example, a number of patients had partial response (PR) or stable disease (SD) (see Table 20).

This combination therapy resulted stable disease (SD) in 1 of 3 patients, establishing anti-cancer activity for this combination.

### Example 14

The effects of 2-acetylnaphtho[2,3-b]furan-4,9-dione in combination with FOLFOX were investigated in adult patients with cholangiocarcinoma (see Table 21).

Patients aged 44-76 who were pretreated with up to 6 lines of prior treatment received oral administration of 2-acetylnaphtho[2,3-b]furan-4,9-dione twice daily together with FOLFOX. For example, 2-acetylnaphtho[2,3-b]furan-4,9-dione was administered at a dose of 240 mg BID or 480 BID in combination with FOLFOX._Oxaliplatin 85 mg/m² together with leucovorin 400 mg/m² were administered intravenously. 5-FU 400 mg/m² bolus was administered intravenously immediately following oxaliplatin/leucovorin infusion, followed by 5-FU 1200 mg/m²/day (total 2400 mg/m² over 46-48 hours) continuous intravenous infusion. This regimen was repeated every 14 days thereafter or until progression of disease, unacceptable toxicity, or other discontinuation criterion was met.

The combination of 2-acetylnaphtho[2,3-b]furan-4,9-dione twice daily together with FOLFOX resulted in stable disease (SD) in a number of patients, establishing anti-cancer activity for this combination.

### Example 15

The effects of 2-acetylnaphtho[2,3-b]furan-4,9-dione in combination with FOLFOX were assessed in adult patients with hepatocellular carcinoma in a clinical study.

Patients aged 64 and 29 who were pretreated with 1 and 4 lines of prior treatment, respectively, received oral administration of 2-acetylnaphtho[2,3-b]furan-4,9-dione twice daily together with FOLFOX. For example, 2-acetylnaphtho[2,3-b]furan-4,9-dione was administered at a dose of 240 mg BID in combination with FOLFOX. Oxaliplatin 85 mg/m² together with leucovorin 400 mg/m² were administered intravenously. 5-FU 400 mg/m² bolus was administered intravenously immediately following oxaliplatin/leucovorin infusion, followed by 5-FU 1200 mg/m²/day (total 2400 mg/m² over 46-48 hours) continuous intravenous infusion. This regimen was repeated every 14 days thereafter or until progression of disease, unacceptable toxicity, or other discontinuation criterion was met.

As shown in Table 10, this combination resulted in stable disease (SD) in both patients, establishing anti-cancer activity for the combination.

TABLE 10
(“BBI608” = 2-acetylnaphtho[2,3-b]furan-4,9-dione)

| | | | | | Prior | |
|---|---|---|---|---|---|---|
| | BBI608 | | | Chemo | # of | Best |
| Patient | (mg) | | Diagnosis | Backbone | Lines | Response |
| 0089 | 240 | bid | Hepatocellular | FOLFOX | 1 | SD; 9% |
| | | | carcinoma | | | regression |
| 0121 | 240 | bid | Hepatocellular | FOLFOX | 4 | SD; 9% |
| | | | carcinoma | | | regression |

### Example 16

Changes in gene expression following treatment with 2-acetylnaphtho[2,3-b]furan-4,9-dione were evaluated using a cancer stem cell PCR array. Numerous molecular markers and genes responsible for cancer stem cell proliferation and self-renewal, such as Nanog, Axl, Atm, and Bmi-1, were found to be downregulated by treatment with 2-acetylnaphtho[2,3-b]furan-4,9-dione (see Table 23).

FaDu sphere cultures were treated for 6 hours with DMSO (control) or 2-acetylnaphtho[2,3-b]furan-4,9-dione at 2 mM. RNA was isolated, reversed transcribed, and the resulting cDNA was analyzed using a qPCR cancer stem cell array. Glyceraldehyde 3-phosphate dehydrogenase (GAPDH) was used as a housekeeping gene to normalize the data. The data in Table 23 shows some of the genes downregulated following treatment with 2-acetylnaphtho[2,3-b]furan-4,9-dione normalized to the control treated sample.

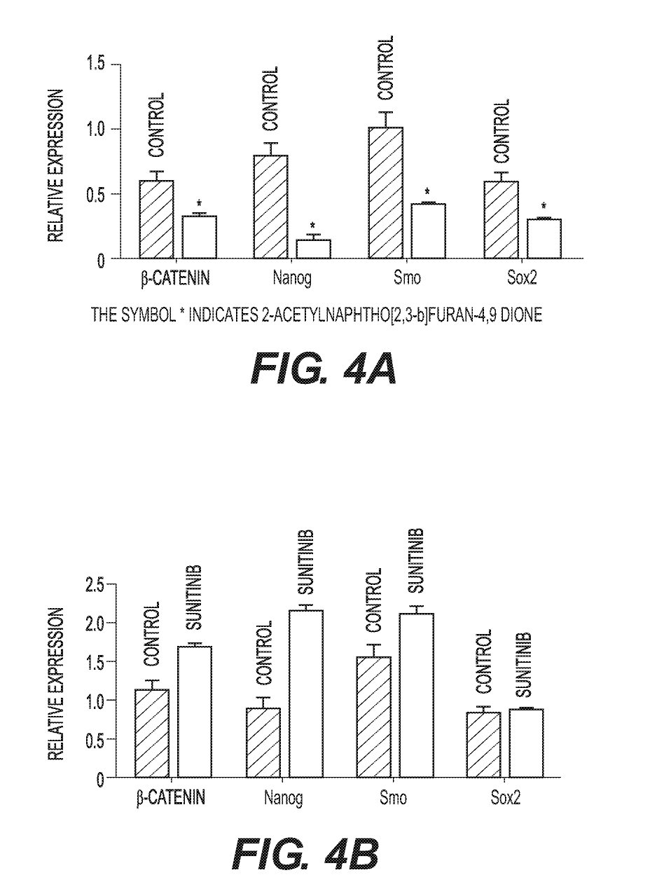

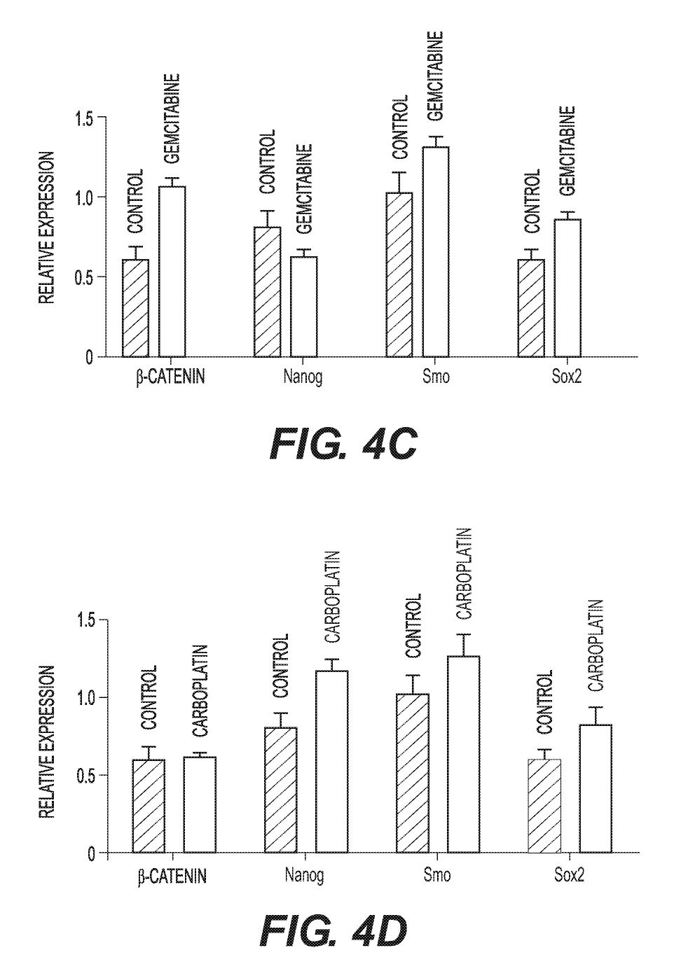

Since treatment with 2-acetylnaphtho[2,3-b]furan-4,9-dione resulted in inhibition of multiple self-renewal pathways, the effect of 2-acetylnaphtho[2,3-b]furan-4,9-dione was next compared with chemo- and targeted therapeutics on stemness gene expression. Treatment of stemness-high cancer cells with 2-acetylnaphtho[2,3-b]furan-4,9-dione resulted in decreased expression of the self-renewal genes β-Catenin, Nanog, Smo, and Sox2. FIGS. 4(A)-(D) depict FaDu cancer stem cells that were treated for 24 hours with DMSO and: (i) 2-acetylnaphtho[2,3-b]furan-4,9-dione (2 mM) (FIG. 4(A)), (ii) sunitinib (20 mM) (FIG. 4(B)), (iii) gemcitabine (2 mM) (FIG. 4(C)), or (iv) carboplatin (32 mM) (FIG. 4(D)). Taken together, these results demonstrated that treatment with 2-acetylnaphtho[2,3-b]furan-4,9-dione decreased expression of molecular markers and genes responsible for cancer stem cell proliferation and self-renewal. The other therapeutics, including sunitinib, gemcitabine, and carboplatin, as well as 5-FU, irinotecan, regorafenib, and oxaliplatin, however, resulted in increased CSC gene expression in certain cases.

TABLE 23

| Gene | % Change | Gene | % Change | Gene | % Change |
|---|---|---|---|---|---|
| NANOG | −93.34 | YAP1 | −47.45 | FLOT2 | −29.19 |
| KLF17 | −90.18 | BMI1 | −47.40 | CHEK1 | −28.88 |
| CD34 | −88.18 | NOTCH1 | −46.24 | B2M | −27.69 |
| LIN28A | −87.52 | ATXN1 | −45.26 | MUC1 | −27.42 |
| POU5F1 | −77.31 | ERBB2 | −43.81 | CD24 | −26.46 |
| PECAM1 | −70.26 | SIRT1 | −43.35 | NFKB1 | −25.54 |
| ATM | −68.65 | WEE1 | −42.86 | ACTB | −24.49 |
| MERTK | −65.78 | FGFR2 | −41.18 | EPCAM | −22.87 |
| NOTCH2 | −64.37 | DDR1 | −38.59 | STAT3 | −22.87 |
| LATS1 | −60.75 | GSK3B | −38.06 | TWIST2 | −21.59 |
| ITGA2 | −60.71 | ENG | −37.62 | PLAUR | −20.64 |
| SMO | −55.56 | DACH1 | −36.55 | EGF | −19.07 |
| TGFBR1 | −53.34 | ALCAM | −36.13 | ALDH1A1 | −18.18 |
| MAML1 | −52.82 | HDAC1 | −36.10 | RPLP0 | −17.48 |
| WWC1 | −51.64 | CD44 | −35.44 | TAZ | −15.09 |
| ITGA6 | −54.63 | HPRT1 | −33.81 | JAG1 | −14.24 |
| ITGB1 | −50.45 | IKBKB | −32.11 | ID1 | −12.68 |
| AXL | −48.50 | DNMT1 | −32.09 | ITGA4 | −11.52 |
| KITLG | −47.99 | ETFA | −31.12 | IL8 | −7.62 |
| JAK2 | −47.94 | FOXP1 | −29.76 | MYC | −3.91 |

The effects of 2-acetylnaphtho[2,3-b]furan-4,9-dione on cancer stem cell markers was also examined in cancer xenograft models with and without irinotecan. Human cancer cells were subcutaneously implanted into the right flank of 5-7 weeks old female athymic nude mice. When tumor size reached 200 mm³, animals were treated with 2-acetylnaphtho[2,3-b]furan-4,9-dione, irinotecan, or a combination of 2-acetylnaphtho[2,3-b]furan-4,9-dione and irinotecan. Tumors were harvested after first dosing.

The harvested tissues were fixed in 3.7% neutral buffered formaldehyde at 4° C. for overnight. The paraffin was embedded, cut to about 5 microns, and affixed onto positively charged slides. After being baked and de-paraffinized, the slides with tumor or control tissues were incubated in 10 mM Sodium Citrate (pH 6.0) for 10 minutes. After antigen retrieval, slides were probed with primary antibodies P-STAT3 (rabbit, Cell Signaling, 1:100), 13-Catenine (mouse, Santa Cruz, 1:400) at 4° C. overnight, and then Alexa Fluor fluorescent dyes-conjugated secondary antibodies (1:500, Invitrogen). After mounting with ProLong mounting medium with DAPI (Invitrogen), the slides were examined under a Zeiss fluorescence microscope with 20× objective, and analyzed with Zen software.

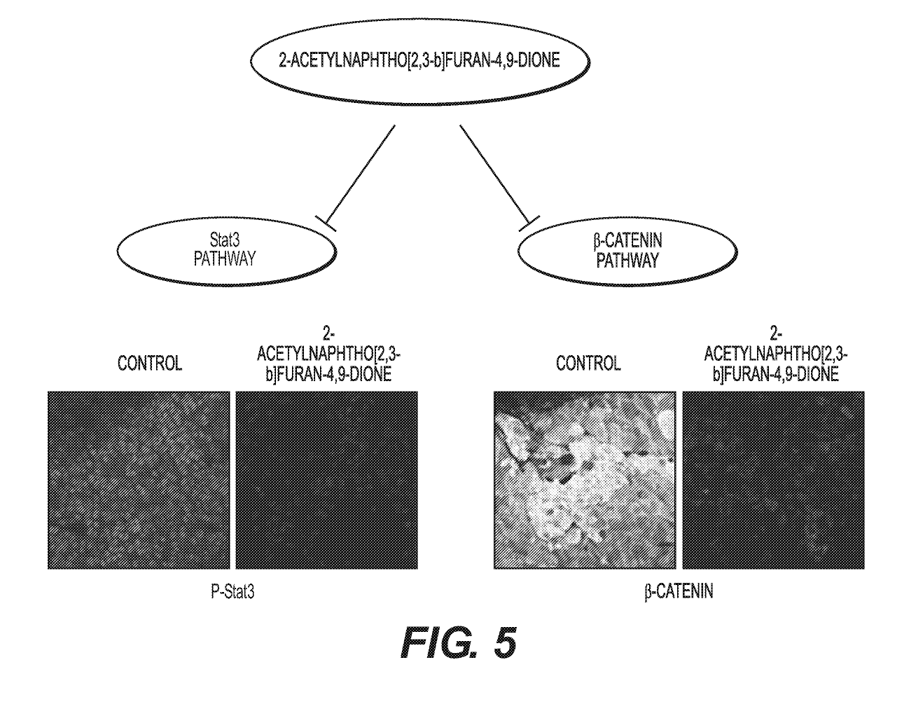

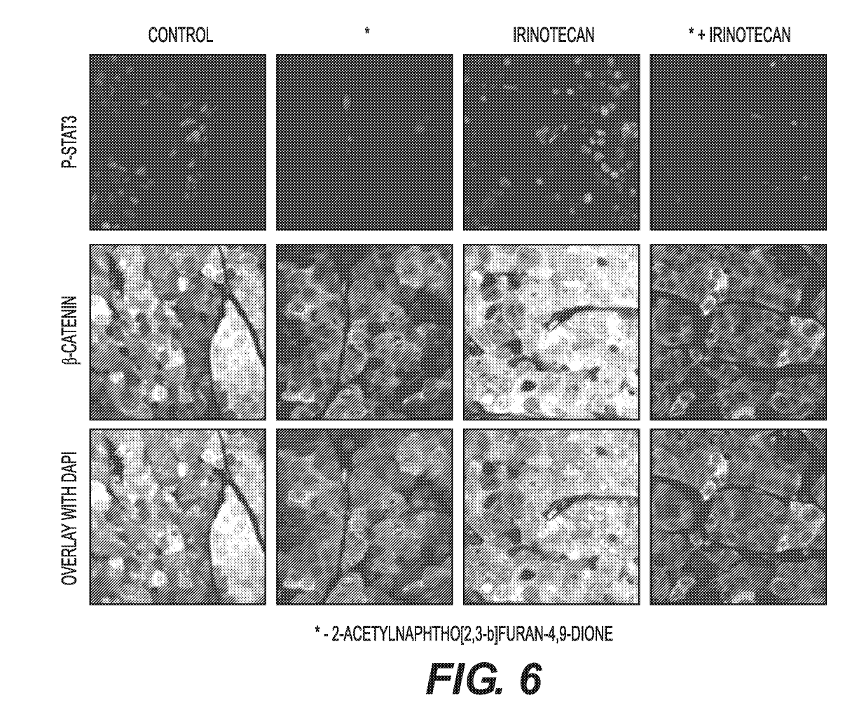

As shown in FIG. 5, 2-acetylnaphtho[2,3-b]furan-4,9-dione alone dramatically reduced expression of both the p-Stat3 and β-catenin stem cell markers. In contrast, as shown in FIG. 6, irinotecan alone resulted in enhanced staining for stem cell markers, which was attenuated by the addition of 2-acetylnaphtho[2,3-b]furan-4,9-dione.

The effects on cancer stem cells of 2-acetylnaphtho[2,3-b]furan-4,9-dione was also examined with and without 5-fluorouracil by using methods similar to those disclosed in U.S. pre-grant Publication No. 2012/0252763, Example 3.

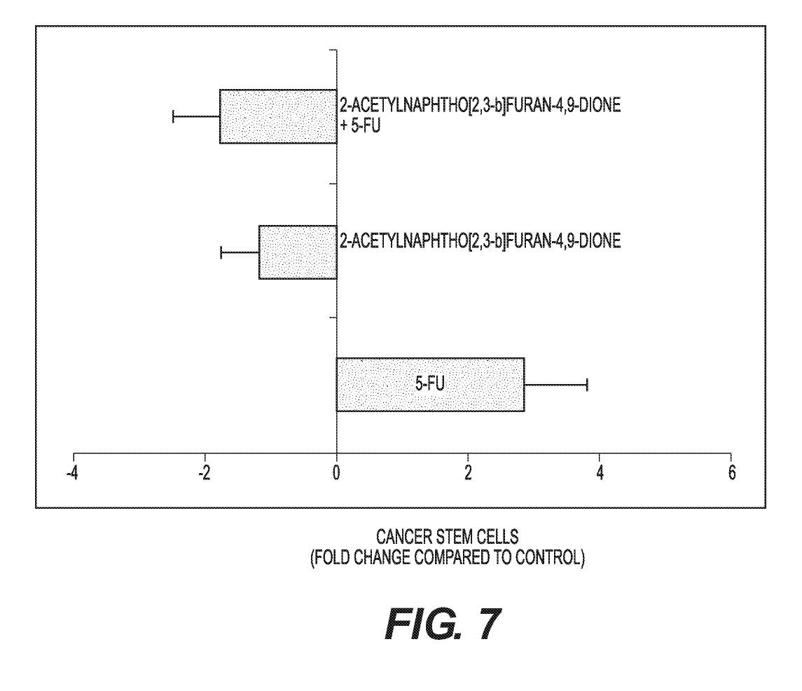

As shown in FIG. 7, 5-fluorouracil alone resulted in a marked increase in the number of cancer stem cells (approximately 3-fold compared to control cells), which was attenuated by the addition of 2-acetylnaphtho[2,3-b]furan-4,9-dione. Moreover, FIG. 7 demonstrated that the effect on cancer stem cells of the combination of 5-fluorouracil and 2-acetylnaphtho[2,3-b]furan-4,9-dione was greater than the added effect of both agents alone. Thus, the combination of 5-fluorouracil and 2-acetylnaphtho[2,3-b]furan-4,9-dione had a greater than additive effect on cancer stem cell growth.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described accordingly, all suitable modifications and equivalents may be resorted to, falling within the scope of the present disclosure.

What is claimed is:

1. A method for treating cancer in a subject comprising administering to a subject whose cancer progressed on at least one prior anti-EGFR therapy:
a therapeutically effective amount of at least one compound of formula (I): 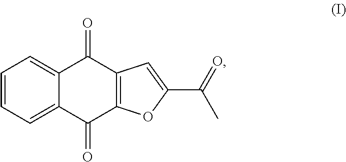 [SMILES: CC(=O)c1cc2c(o1)C(=O)c1ccccc1C2=O]
and
a therapeutically effective amount of at least one panitumumab compound chosen from panitumumab, pharmaceutically acceptable salts thereof, and solvates of any of the foregoing or
a therapeutically effective amount of at least one cetuximab compound chosen from cetuximab, pharmaceutically acceptable salts thereof, and solvates of any of the foregoing.

2. A method for resensitizing a subject to anti-EGFR therapy comprising administering to a subject whose cancer progressed on at least one anti-EGFR therapy:
a therapeutically effective amount of at least one compound of formula (I):

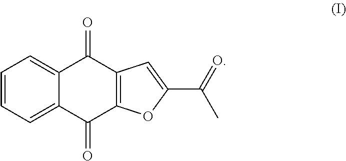 [SMILES: CC(=O)c1cc2c(o1)C(=O)c1ccccc1C2=O]

3. A method of simultaneously
(i) inhibiting, reducing, and/or diminishing survival and/or self-renewal cancer stem cells and
(ii) inhibiting, reducing, and/or diminishing survival and/or proliferation of heterogeneous cancer cells
in a subject comprising administering to a subject in need thereof
a therapeutically effective amount of at least one compound of formula (I): 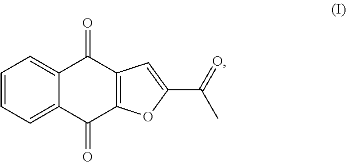 [SMILES: CC(=O)c1cc2c(o1)C(=O)c1ccccc1C2=O]
and
(a) a therapeutically effective amount of at least one panitumumab compound chosen from panitumumab, pharmaceutically acceptable salts thereof, and solvates of any of the foregoing;
(b) a therapeutically effective amount of at least one cetuximab compound chosen from cetuximab, pharmaceutically acceptable salts thereof, and solvates of any of the foregoing;
(c) a therapeutically effective amount of at least one capecitabine compound chosen from capecitabine, pharmaceutically acceptable salts thereof, and solvates of any of the foregoing;
(d) a therapeutically effective amount of at least one regorafenib compound chosen from regorafenib, pharmaceutically acceptable salts thereof, and solvates of any of the foregoing; or
(e) a therapeutically effective regimen of FOLFOX.

4. A method for treating cancer in a subject comprising administering to a subject whose cancer progressed on at least one prior capecitabine therapy:
a therapeutically effective amount of at least one compound of formula (I): 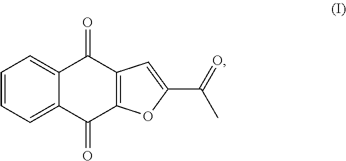 [SMILES: CC(=O)c1cc2c(o1)C(=O)c1ccccc1C2=O]
and
a therapeutically effective amount of at least one capecitabine compound chosen from capecitabine, pharmaceutically acceptable salts thereof, and solvates of any of the foregoing.

5. A method for resensitizing a subject to capecitabine therapy comprising administering to a subject whose cancer progressed on at least one prior capecitabine therapy
a therapeutically effective amount of at least one compound of formula (I):

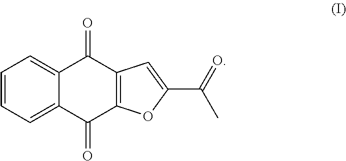 [SMILES: CC(=O)c1cc2c(o1)C(=O)c1ccccc1C2=O]

6. A method for treating cancer in a subject comprising administering to a subject whose cancer progressed on at least one prior regorafenib therapy
a therapeutically effective amount of at least one compound of formula (I): 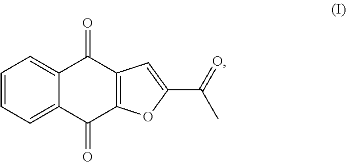 [SMILES: CC(=O)c1cc2c(o1)C(=O)c1ccccc1C2=O]
and
a therapeutically effective amount of at least one regorafenib compound chosen from regorafenib, pharmaceutically acceptable salts thereof, and solvates of any of the foregoing.

7. A method for resensitizing a subject to regorafenib therapy comprising administering to a subject whose cancer progressed on at least one prior regorafenib therapy
a therapeutically effective amount of at least one compound of formula (I):

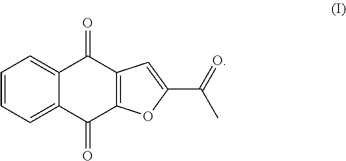 [SMILES: CC(=O)c1cc2c(o1)C(=O)c1ccccc1C2=O]

8. A method for treating cancer in a subject comprising administering to a subject whose cancer progressed on at least one prior FOLFOX therapy
a therapeutically effective amount of at least one compound of formula (I): 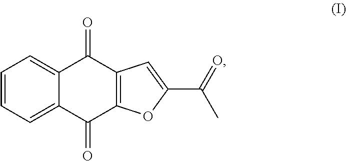 [SMILES: CC(=O)c1cc2c(o1)C(=O)c1ccccc1C2=O]
and
a therapeutically effective regimen of FOLFOX.

9. A method for resensitizing a subject to FOLFOX therapy comprising administering to a subject whose cancer progressed on at least one prior FOLFOX therapy
a therapeutically effective amount of at least one compound of formula (I):

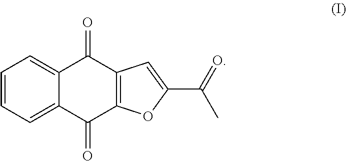 [SMILES: CC(=O)c1cc2c(o1)C(=O)c1ccccc1C2=O]

10. The method according to any one of claims 1-9 and 23-24, wherein the at least one compound of formula (I) is chosen from compounds having formula (I) 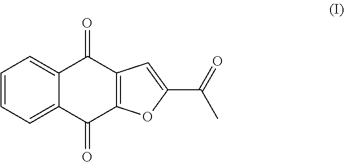 [SMILES: CC(=O)c1cc2c(o1)C(=O)c1ccccc1C2=O]
prodrugs, derivatives, pharmaceutically acceptable salts of any of the foregoing, and solvates of any of the foregoing.

11. The method according to claim 3 or 23, wherein the at least one compound of formula (I) is administered with a therapeutically effective amount of at least one capecitabine compound chosen from capecitabine, pharmaceutically acceptable salts thereof, and solvates of any of the foregoing, and a therapeutically effective amount of at least one oxaliplatin compound chosen from oxaliplatin, pharmaceutically acceptable salts thereof, and solvates of any of the foregoing.

12. The method according to claim 4, further comprising administering a therapeutically effective amount of at least one oxaliplatin compound chosen from oxaliplatin, pharmaceutically acceptable salts thereof, and solvates of any of the foregoing.

13. The method according to claim 3 or 23, wherein the at least one compound of formula (I) is administered with a therapeutically effective regimen of FOLFOX and a therapeutically effective amount of at least one angiogenesis inhibitor.

14. The method according to claim 8, further comprising administering a therapeutically effective amount of at least one angiogenesis inhibitor.

15. The method according to claim 13 or 14, wherein the at least one angiogenesis inhibitor is chosen from bevacizumab, pharmaceutically acceptable salts thereof, and solvates of any of the foregoing.

16. The method according to any one of claim 1, 2, or 4-9, wherein the subject's cancer is associated with an aberrant Stat3 pathway.

17. The method according to claim 3, 23, or 24, wherein the differentiated tumor cells are from a cancer associated with an aberrant Stat3 pathway.

18. The method according to claim 16 or 17, wherein the cancer associated with an aberrant Stat 3 pathway is chosen from colon adenocarcinoma, rectal adenocarcinoma, gastroesophageal junction adenocarcinoma, gastric adenocarcinoma, pancreatic adenocarcinoma, esophageal adenocarcinoma, cholangiocarcinoma, hepatocellular carcinoma, and colorectal cancer.

19. The method according to claim 18, wherein the cancer associated with an aberrant Stat 3 pathway is advanced, metastatic, unresectable, or recurrent.

20. The method according to any one of claims 1-9 and 23-24, wherein the at least one compound of formula (I) is administered at a dose of about 480 mg, about 960 mg, or about 1000 mg per day.

21. The method according to claim 20, wherein the at least one compound of formula (I) is administered in a divided dose.

22. The method according to an one of claims 1-9 and 23-24, wherein the at least one compound of formula (I) is administered at a dose of about 240 mg twice daily, about 480 mg twice daily, or about 500 mg twice daily.

23. A method of treating cancer in a subject comprising administering to a subject in need thereof
a therapeutically effective amount of at least one compound of formula (I): 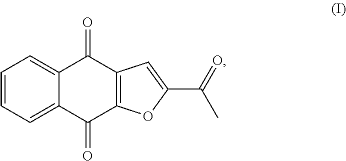 [SMILES: CC(=O)c1cc2c(o1)C(=O)c1ccccc1C2=O]
and
(a) a therapeutically effective amount of at least one panitumumab compound chosen from panitumumab, pharmaceutically acceptable salts thereof, and solvates of any of the foregoing;
(b) a therapeutically effective amount of at least one cetuximab compound chosen from cetuximab, pharmaceutically acceptable salts thereof, and solvates of any of the foregoing;
(c) a therapeutically effective amount of at least one capecitabine compound chosen from capecitabine, pharmaceutically acceptable salts thereof, and solvates of any of the foregoing;
(d) a therapeutically effective amount of at least one regorafenib compound chosen from regorafenib, pharmaceutically acceptable salts thereof, and solvates of any of the foregoing; or
(e) a therapeutically effective regimen of FOLFOX.

24. The method according to claim 23, wherein the cancer is cancer is advanced, metastatic, unresectable, recurrent, or/and refractory
